(19) United States
(12) Patent Application Publication
(10) Pub. No.: US 2021/0238698 A1
(43) Pub. Date: Aug. 5, 2021

(54) METHODS OF DIAGNOSING AND TREATING CANCER PATIENTS EXPRESSING HIGH LEVELS OF TGF-B RESPONSE SIGNATURE

(71) Applicant: MEDPACTO, INC., Seoul (KR)

(72) Inventors: Chan-young OCK, Seoul (KR); Seong Jin KIM, Seoul (KR)

(73) Assignee: MEDPACTO, INC., Seoul (KR)

(21) Appl. No.: 17/255,432

(22) PCT Filed: Jun. 29, 2019
(86) PCT No.: PCT/IB2019/055475

§ 371 Date: Dec. 23, 2020

Related U.S. Application Data
(60) Provisional application No. 62/690,567, filed on Jun. 27, 2018.

(51) Int. Cl. C12Q 1/6886; A61K 31/437; A61K 39/395; A61P 35/00

(52) U.S. Cl. CPC C12Q 1/6886; A61K 31/437; C12Q 2600/106; A61P 35/00; C12Q 2600/158; A61K 39/3955

(57) ABSTRACT

The present invention relates to methods for treating colorectal cancer patients comprising selecting patients who express high level of the TGF-β response signatures and co-administering TGF-β inhibitor with human immunoglobulin. The invention also relates to the method of diagnosing and selecting patients which is likely to benefit from the treatment comprising the determination of the gene expression levels of TGF-β response signature by analyzing histopathologic images of stroma or RNA sequence from the patients.

### CROSS REFERENCE TO RELATED APPLICATION

This application claims the benefit of U.S. provisional patent application Ser. No. 62/690,567, filed Jun. 27, 2018, the contents of which are incorporated herein by reference.

### FIELD OF THE INVENTION

The present invention relates to methods for treating cancer patients expressing high levels of TGF-β response signatures by co-administering TGF-β inhibitor with human immunoglobulin. Another embodiment of the invention relates to the method of diagnosing a patient which is likely to benefit from the treatment by analyzing gene expression level of TGF-β response signatures and treating the subject.

### BACKGROUND OF THE INVENTION

Metastasis refers to the spread of cancer cells from the initial place to a different part of the body. Treatment of any form of cancer is dependent on the presence of metastasis. If the cancer spreads to other places, it may also decrease the survival of the patient.

Previously, the overexpression or under expression of certain genes has been shown to be related to the propensity of tumors to metastasize to the lungs or bones. In some cases, the overexpression of certain genes has been linked to the production of, or responsiveness to, certain mediators that can influence the tumor cells and confer on them the ability to seed other organs and survive there. The microenvironment of the tumor, including the presence of cytokines, growth factors and proteases, could influence the ability of tumor cells to metastasize. The cytokine TGF-β has been implicated in the modulation of tumor progression in various experimental systems. Low expression levels of TGF-β receptor in estrogen receptor negative tumors is associated with better overall outcome, whereas overexpression of TGF-β is associated with a high incidence of distant metastasis.

TGF-β response signature (TBRS) is gene expression signature which are directly induced by TGF-β gene responses. Previous studies described the correlations between TGF-β, TBRS, cancer metastasis and cancer relapse. For example, WO 201004228 describes the identification of 176 genes, the expression levels of which correlate with the prognosis of colorectal cancer. Padua et al. (Cell, 133(1): 66-77, 2008) describes TBRS associates with breast cancer metastasis and Calon et al. (Cancer Cell, 22, 571-584, 2012) reports that TBRS in stromal cells predicts metastasis and colorectal cancer relapse. Furthermore, Tsushima et al. (Clin Cancer Res., 7(5):1258-62, 2001) showed high levels of TGF-β in the serum of the colorectal cancer patients are associated with liver metastasis and poor clinical outcome. The contents of WO 201004228, Padua et al., Calon et al., and Tsushima et al. are incorporated herein by reference by their entireties.

### SUMMARY OF INVENTION

The present invention provides the method of treating a subject with cancer comprising the steps of measuring whether the patient expresses high levels of TGF-β response signatures in fibroblasts and of co-administering an effective amount of TGF-β inhibitor with human immunoglobulin to the subject.

Another aspect of the present invention provides the method of diagnosing gene expression levels of TGF-β response signature of a subject with cancer by analysis of histopathologic image of stroma or RNA sequence obtained from the subject. Another aspect of the present invention provides the method of diagnosing gene expression levels of TGF-β response signature of a subject with cancer by analysis of histopathologic image of stroma or RNA sequence obtained from the subject and treating the subject. Another aspect of the present invention provides a new TGF-β response signature comprising a subset of 11 genes, i.e., SERPINE1(PAI1), GADD45B, TIMP3, LMCD1, PLAUR, IL6, NUAK1, DACT1, EPHA4, SNAI1, and MEOX1.

### BRIEF DESCRIPTION OF THE DRAWINGS

FIG. 1 is a table showing the RNAseq data and tumor assessment data analyzed from the tumor biopsy samples.

FIG. 2 is a graph showing the anti-tumor activity of Vactosertib (the compound represented by Formula II; TEW-7197).

FIG. 3 is a graph showing the analysis results of F-TBRS and cytolytic score in cancer of Phase I responders and non-responders.

FIG. 4 is a graph showing the analysis results of the EMT (epithelial-mesenchymal transition)/F-TBRS expression distribution from the TCGA (The Cancer Genome) database of bladder cancer.

### DETAILED DESCRIPTION OF THE INVENTION

The present invention provides the method of treating cancer patients comprising the steps of determining whether the patient expresses high levels of TGF-β response signatures in fibroblasts and of co-administering TGF-β inhibitor with human immunoglobulin.

Another aspect of the present invention provides the method of diagnosing gene expression levels of TGF-β response signature of patients by analysis of histopathologic image of stroma or RNA sequence obtained from the patients. In an embodiment of the present invention, the method of diagnosing gene expression levels of TGF-β response signature of patients by analysis of histopathologic image of stroma or RNA sequence obtained from the patients and treating the patients is provided. In an alternative embodiment of the present invention, the assay of using the methods, such as quantitative-PCR or real-time PCR, to analyze a smaller subset of genes may be employed.

In an embodiment of the present invention, the invention relates the method of treating cancer patients comprising the steps of determining whether the patient expresses high levels of TGF-β response signatures in fibroblasts, T-cells, macrophages and endothelial cells; and when the patient has high levels of TGF-β response signatures in cells that comprise the tumor microenvironment (TME), which includes but is not limited to fibroblasts, T-cells, macrophages and endothelial cells, then co-administering TGF-β inhibitor with human immunoglobulin. In another embodiment of the present invention, the invention relates the method of treating a cancer patient comprising the steps of identifying the patient expressing high levels of TGF-β response signatures in cells that comprise the tumor microenvironment (TME), which includes but is not limited to fibroblasts, T-cells, macrophages and endothelial cells, and of co-administering TGF-β inhibitor with human immunoglobulin.

Another aspect of the present invention provides Vactosertib TGF-β response signature (VRS) comprising a subset of 11 genes, i.e., SERPINE1(PAI1), GADD45B, TIMP3, LMCD1, PLAUR, IL6, NUAK1, DACT1, EPHA4, SNAI1, and MEOX1.

As used herein the cancer includes, but not limited to, colorectal cancer, melanoma, breast cancer, bladder cancer, colon cancer, kidney cancer, lung cancer, ovary cancer, pancreas cancer, prostate cancer, rectal cancer, stomach cancer, thyroid cancer, uterus cancer and other types of cancer. Preferably, cancer is colorectal cancer, melanoma or breast cancer. Most preferably, cancer is colorectal cancer.

### TGF-β

Transforming growth factor (TGF)-β is a cytokine that regulates cell proliferation and differentiation, wound healing, extracellular matrix production, or the like. TGF-β family belongs to TGF-β superfamily, and this TGF-β superfamily includes activins, inhibins, bone morphogenetic proteins, and anti-Mullerian hormone. The tumor cells and the stromal cells within the tumors in late stages of various cancers generally overexpress TGF-β. TGF-β would lead to stimulation of angiogenesis and cell motility, suppression of the immune system, and increased interaction of tumor cells with the extracellular matrix. TGF-β receptors are serine/threonine kinase receptors, and they are divided into TGF-β receptor 1, TGF-3 receptor 2, and TGF-β receptor 3. Of them, TGF-β receptor 1 is also called an activin A receptor type II-like kinase (ALK5).

Accordingly, for effective prevention or treatment of cancer, there is a need for a pharmaceutical composition capable of effectively inhibiting a TGF-β signaling pathway as well as improving antitumor immunity.

### TGF-β Response Signature

The TGF-β response signature of Fibroblast (F-TBRS) is selected from FLT1, COL10A1, IGFBP3, NOX4, MEX3B, GAS1, INHBA, VEGFA, CDKN2B, NA, FBXO32, CALB2, CTGF, KANK4, NET1, HEY1, SERPINE1, ESM1, TIMP3, SYNE1, BHLHE40, PLAUR, APBB2, FGF1, ANGPTL4, LMCD1, PGM2L1, KAL1, TNFAIP6, OSGIN2, PODXL, PCDH9, C13orf33, RASGRP3, LOH3CR2A, SPSB1, FN1, GADD45B, TRIB1, STK17B, KLF7, LRRC8C, FNIP2, TGFB2, FRMD4A, CNTN1, NGF, NUAK1, EFNB2, TSPAN2, CHST11, EGR2, DAAM1, PALLD, GPR161, CALD1, LOC728449, MGC16121, HIVEP2, RHOU, KDM6B, FOXP1, ELMOD1, SEMA7A, PTHLH, PMEPA1, HAS2, SNORD114-3, GRB14, LIF, TSHZ3, PDLIM4, LOC728264, ZNF365, PDGFC, JUNB, CILP, BPGM, ARHGEF3, PGBD5, TAGLN3, TUFT1, GPR183, S1PR5, CLDN4, MBOAT2, CNNM4, DNAJB5, C3orf52, DHRS2, SOX4, EPHA4, COL27A1, SMAD7, F2RL1, LOC100128178, RASL12, SLC35F2, SETBP1, DOCK10, C5orf13, DNAJC18, DACT1, WNT9A, ETV6, FGF18, HBEGF, TNC, SDC1, KIAA1755, EDN1, ITGB6, MURC, PTGS2, PLEK2, LOC201651, STEAP2, SLC46A3, SNX30, RYBP, TMEM49, SORBS2, HIC1, NEDD9, ARHGEF40, IFIH1, GZMK, VEPH1, PIK3CD, IL6, YIPF5, SKIL, RASD1, JARID2, IL11, SNAI1, SOX6, STK38L, NKX3-1, CDH6, PELI1, PRDM1, PDPN, WNT2, LMO4, C4orf26, CACHD1, PRR5L, TMEM2, DDX10, MTSS1, CLDN14, JHDM1D, SLC19A2, PLCE1, PRR9, MEGF9, GOPC, MSC, PPP1R14C, PKNOX2, MSX2, SNCAIP, SLC35F3, LOC727930, HS3ST3B1, MEOX1, E2F7, AUTS2, FUT4, DLX2 or TBX3.

The TGF-β response signature of T-Cell (T-TBRS) is selected from TIMP1, RAB31, CLIC4, MXRA7, SERPINE1, INPP5F, GEM, ANXA5, LMCD1, CST6, RBPJ, RASGRP3, PLAU, LOH3CR2A, ITGAV, MAP4, KLF7, ABHD2, BMP1, KCNK1, RGS16, ATXN1, NMB, EGR2, IL1RN, KLF10, APOD, PIK3IP1, CRIP1, SLC5A3, NR4A3, EVI2A, FAM102A, TIAM1, ALOX5AP, ZNF365, TNFSF4, PPAP2A, PKIA, FBXO11, DCLRE1C, SOX4, C18orf1, NPTX1, CSGALNACT1, TMOD1, ATP1B1, CCR4, CD83, LRIG1, ADO, LRBA, DIXDC1, SYNJ2, RIMS3, JUN, CD96, PRKCD, S100PBP, HLF, ABCC4, CXCL13, PPARG, AHCYL2, AQP3, TSPAN13, SLC1A4, TFEB, ITGAE, APOBEC3G, CDYL, IL7R, IL9, ZNF238, MYH6, GPR35 or MB.

The TGF-β response signature of Macrophage (M-TBRS) is selected from C5orf23, RAI14, NA, AHNAK2, ZNF532, CEP170, POSTN, PALM2-AKAP2, SPP1, VEGFA, ERRFI1, APOE, FBXO32, PCOLCE2, EHD2, ENO2, KCNJ8, PLAUR, TANC2, VCAN, CD109, CDKN1C, PLA2G16, AXL, KANK4, PLOD2, ULBP2, RASSF8, TBC1D8, SERPINE1, SOX11, DOCK4, SULF1, IER5L, C15orf52, FGD6, PDLIM7, EBF1, AMIGO2, PTPRM, BHLHE40, BACH2, OLFML2B, APBB2, MFGE8, MYADM, SLC16A6, ACTN1, OLR1, DPP4, TRNP1, IGFBP4, B3GALNT1, PCDHB2, ABLIM3, LMTK3, HTRA1, CLDN11, TMCC3, DPYSL3, ADAMTS2, LOC641518, MYH10, CST6, SKI, ELK3, PHLDA1, KAL1, CCDC88A, CYTH3, ATP10A, ITGA5, SLC43A3, ZNF281, ADAM12, HSPG2, MSR1, LAMC1, APOC1, PDLIM4, VSIG10L, PACSIN3, LARP6, THBS1, CXCR7, GABARAPL1, SLC11A1, GADD45B, BTG1, MMP8, ITGAV, SPHK1, GDF5, ABHD2, HPGD, KIAA1217, PECAM1, PDE4DIP, FLCN, THBS3, CXCR4, GPX3, TM6SF1, DLC1, FKBP1B, S100A2, BEAN, NMB, NAV1, TREM1, PDK4, MEF2A, ST3GAL6, CDK14, SPTBN1, CAMK2N1, RUNX2, SLIT3, USP46, LRRC32, ANGPT2, FADS3, ADAMS, CD151, RGS16, SLC44A2, GULP1, MID2, LYNX1, COL22A1, BNC2, SDCCAG8, ORAI2, MMP19, DENND3, ZEB1, DNASE2, ADAMS, FST, IL1RAP, AZI2, SOCS6, MRC2, UBXN2B, HTRA4, SPOCD1, ADARB1, ANO4, DFNB31, RGS1, SDS, DCLK1, OBSL1, TMEM180, C21orf7, GNG11, CCL7, UPP1, FZD4, FCGR1B, DPH3, N4BP2L2, GRAMD1B, GLI3, FBLN5, KLF10, CLEC5A, MAMLD1, TCEAL3, SEMA4B, IRS1, GPC1, WBP5, MYOZ1, FPR3, PROC, FNDC3B, FZD10, MRO, ADM, PLXDC2, MBD6, LDLRAD3, SLC22A4, SH2D3C, KCNK10, FRY, PMEPA1, RIN3, MOAP1, TMEM223, DIO3, TTC6, IL1RL1, DIO3OS, CPE, FAM89A, CD300LF, SLC29A1, FMNL2, MTUS1, GRB10, FAM110B, LOC646903, CRTAC1, C5orf62, DOCK9, TLR7, NMBR, ALOX5, DNM3, LHFPL2, ITGA2, FGF18, IFIT2, TGFBR1, BTC, TANC1, CDK5RAP2, FLRT2, FAM27E3, MGC24103, TSPAN2, SLC31A2, SPRY1, TNFRSF12A, RGL3, FHL3, LOC283033, LDB2, ATP11B, DAPK1, GAL3ST4, TMEM163, ABCD1, LTB4R, SLC6A8, ALOX5AP, MASP1, TMEM51, XRCC6BP1, RNF17, LAIR1, ZNF618, ADAM17, DUSP1, DHX58, NCEH1, SLC22A15, NCOR2, ZNF555, HOXC10, ARL9, TSKU, SPATA12, C1orf204, CHD3, IQCG, LOC100128501, SLFN5, CCDC40, S100A11, MAPK8IP3, TM7SF4, AICDA, PLEKHA7, C1orf106, UNC5C, MYOZ3, ADCY2, KCNH3, IL28RA, JAG1, MAP3K2, COL8A2, TPST1, FGF7, DKK1, PRRG4, PPARD, GPR157, AREG, FER1L4, UNC5B, LOC100240734, RP1L1, NUPR1, PKIA, IQCH, WRNIP1, MCOLN3, TTC7A, RAB23, PHF17, EVX1, FPR1, AQP9, SIM1, CACNA1G, NFKBIL2, FAM84B, FAM7A3, PEX14, MBOAT7, GUCA1A, RUNX1, BEST1, SPAM1, ADAMTS18, HCFC1R1, PPOX, STOX2, SPATS2L, TOP1P2, ARL4A, VSIG1, P4HA2, KCP, TRIM55, LOC283050, RHOB, DISP1, MMP14, MMP7, ABCG2, ABHD8, ARL5A, G0S2, CMTM1, FFAR1, MYH8, TES, SEMA6B, CES1, SLC10A2, KLHL6, LAPTM5, CLDN12, GPR26, LOC151438, C1orf150, PEX2, FAM113B, FABP6, SNTB1, LIMS1, KLF15, SOAT1, SH2D1B, RNASEH2B, MET, SLC39A12, KRT13, RAB3B, MAN2A2, CNBD1, VASH1, SEC22C, UBQLNL, SUCLG1, ZFYVE19, RIF1, RHOBTB2, UNC80, ARMC8, CADM2, C3, SLC8A1, ABCC3, DPY19L1, TSPAN5, AAA1, SLC26A10, SMAD7, HAMP, MERTK, TRIM62, TP53I11, CLDND2, SLC16A10, AMPH, ZNF687, CNTNAP3B, TPM1, GPX7, NCRNA00113, MTHFD1L, NOS3, LOC286437, SLC24A2, RNF6, SERINC2, PSME4, C8orf55, TSPAN3, CADM1, SLC16A3, LOC154822, TFAP2C, FAM40B, IL3RA, IFIT3, SH3GL2, GLCCI1, KRTAP11-1, C1orf91, EPAS1, OR10D3, ASIP, TDRD9, YPEL2, MIA3, BTBD19, GPR84, PHC2, MGST1, MFAP3L, NR4A2, GLUL, TLE3, MAPKAPK2, DDA1, TPD52L1, FOXD4, GNB4, LSR, SMURF1, C2orf40, FKRP, CLCN4, OSM, HECW1, DIRAS2, CLVS1, MME, ZNF549, DEC1, SKIL, SS18, DGCR6L, RWDD2A, MBD4, RUNX1T1, PXN, LOC283104, ECE1, FZD7, RNASE4, PRIM2, BTBD11, TLX3, HP, ZNF589, TRIO, PRPF40B, SOX18, DDHD1, CLPS, ERMP1, SLC16A8, CDKN1A, AK7, STX6, PHYHD1, PCBP3, OLFML3, ZBTB16, C9orf117, SOX4, PCDHGB8P, EYA2, TMTC1, EN1, ELL2, SLC45A3, HRG, DENND5B, MDFIC, TGM2, GPD2, HBEGF, AKR1C2, GLDN, OCIAD2, LONRF3, MAP9, LOC283454, NTRK2, SLC6A12, IL18RAP, PLEKHA5, NT5DC4, CPD, ANKRD29, PCMTD1, FAM178B, ZFP36L1, SMURF2, SIGLEC8, LOC284454, LOC401097, C17orf91, NLRP12, USP53, C19orf59, NPR1, FMNL3, SLC41A1, KCNA3, CACNA2D1, ARID3B, GAPT, BLOC1S2, IGF2BP1, PCNX, SCD, CCDC157, BLMH, SLC5A7, MFI2, FKBP15, TRMT1, FCGR3B, TEX14, OK/SW-CL.36, COX15, MEX3D, LTA4H, AKR1C1, CNIH4, NPTXR, TP53INP2, C12orf59, KLHL29, LARGE, MMP2, KCNC3, NAPSB, ACADVL, C1orf126, CYP19A1, TNNT2, NRK, MSTN, CYLD, RGS8, HDGFRP3, HS3ST5, LOC255167, KCNIP2, IL17RB, ANG, FLJ13197, TPSB2, FURIN, NEK6, CD58, PYROXD1, ALK, GOLT1A, CNKSR3, CHD9, RHOH, L3MBTL4, HAL, S100A8, NLRC4, PTGER3, CLP1, SLC35F4, IL27RA, PARVA, CARD11, MTMR11, FSTL5, CSN1S1, ANK1, BCAS4, PPIH, DIXDC1, FOS, MIA2, FOXQ1, MNDA, SLC1A2, KIAA1274, COQ2, WLS, MSRA, NR4A3, APOC2, GSN, TFAP2B, CCNA1, LRRC1, PRDM14, SMAD6, ST8SIA2, PHGDH, TMEM111, PSEN1, HIPK2, ARPP21, SBNO1, SP100, NEDD9, PKD2L1, FBP1, TTC12, FAM71F2, TOPORS, B3GNT5, GPR25, EWSR1, ADC, FBXO9, DYX1C1, CELF6, TMEM38B, TMED5, ZNF527, MEX3C, LRRC27, GCHFR, PGLYRP2, LMX1A, GPR34, CADM4, RIT2, UTY, ARFGEF2, NUP62CL, PDCD1, PTPDC1, PWRN2, PTPRZ1, CDK3, RBP4, PLAC1L, C12orf49, LOC339807, DYNC1H1, LRRC6, EFCAB5, SLC46A2, NCR3, TCEB3B, TUBE1, FCRL1, GPR37, RALA, SYTL1, NKTR, CES4, CAMP, DDX52, EPB41L5, FOXG1, GOLGA8IP, FLJ33996, GGT7, SLC35D2, TCTN2, MYO6, SLC44A5, ATP13A3, LIPG, DENR, ZNF540, TPTE2P1, POU3F1, BOLL, A2ML1, SH2D4A, LOC100287525, SERPINB11, TBC1D10C, IL18BP, SDC1, GPR110, TPSAB1, SSH2, C14orf109, DNAL1, NDP, FGFRL1, XPO1, MKI67IP, SLC7A1, ALDH1A1, PDXK, RDH12, DNMT3B, CYP2W1, SYT2, CLIP3, C9orf9, DDX28, NSFL1C, SPDEF, AGAP3, CCDC123, KLF12, SPINT1, MIR155HG, DDX3X, LOC158696, ID3, LOC728431, B3GNT7, MAB21L1, TGIF1, FAM82A2, LOC100133131, RNFT1, LCK, IL1A, MXD1, REPS2, C11orf75, MLPH, CCDC63, ENO3, RABGAP1L, SORL1, PHACTR1, ERBB3, C11orf63, LOC731157, CCDC114, CHERP, ROCK1, CPEB3, TYRO3, CRX, FARP1, ESR2, ISM1, UBE2E1, BLID, WNT4, PTPN22, LOC285423, LRPAP1, TNS1, RALGPS2, SEMA7A, ADAMTS19, C17orf66, SNAI3, DSTN, STOM, KCNIP4, TACC2, SLC25A30, CXorf31, TP63, SLC5A12, RRAD, HAPLN1, LTBR, SYT14, TNFRSF10D, MARCH3, CCDC68, AK1, CALHM3, MAP4K2, MAP1LC3A, IL1R2, KIAA0317, SCGN, CNTNAP3, AGPAT2, GNRHR, AGRP, HES1, DMD, MTMR3, EML4, DNASE1, PNKD, ACER3, NPAS1, PSEN2, MAPK8, LOC553137, UTRN, BUB1, DCUN1D3, C8orf8, CDH1, IGKC, EDNRB, CDS1, GATA4, WIPI1, PHTF1, FTCD, ZNF79, SH3RF3, C21orf67, FLJ30375, ZFP41, GLRX, C10orf95, ID2B, LOC541473, C6orf195, FNDC5, WDR52, SPINK5, LOC642587, RGS12, TGM5, CYGB, SYT5, LOC389634, LOC121952, DEFB124, CYB5A, MYO5BP2, P2RY11, RNF183, ABHD5, C15orf62, TRIM2, LOC643837, PRSS21, SLC6A4, TBC1D16, ICK, AQP3, OR51E1, MINK1, ZNF800, OLIG2, UNC13C, RNF166, LOC729178, ST18, PDPN, PPP2R2C, GAD2, KCNK13, C14orf56, KLK2, LRRC43, TRAIP, TSPAN13, CACNA1E, FAM71B, BPY2, DBF4B, SYTL3, CYP2F1, OR1J4, GBP6, PLCG1, LIPJ, TPD52, KLHL11, C9orf44, LRRC37BP1, TMEM207, TFEB, TRIM36, SLCO2B1, PRRSL, PPM1H, CLDN23, TRPM3, AMBRA1, SLC31A1, ASB15, NKG7, C20orf7, C12orf48, C18orf8, SNX7, LHX6, USP22, TLR3, TASP1, SNX10, SLC4A11, RAPGEF3, TFCP2L1, APOC4, HOXB8, NEB, QPCT, SGCB, DUSP16, ICOSLG, CAPN5, EHF, ENOPH1, DGAT2, TIAM2, INADL, N4BP2L1, LOC149773, TFR2, HN1, SLC23A3, LOC646576, CYP27A1, KIAA1244, HPS4, PNPLA7, CKLF, GPR125, PTH2R, ESR1, PAQR5, ENG, DNAJC6, AACSL, OR4D2, ATP2B2, PARP10, SLC24A1, LFNG, CYP4Z2P, ABCG1, EPB41, SLC6A15, PKD1L2, KLF14, ARHGAP11A, LOC441461, RHEB, SRRM3, SERHL2, POPDC3, APOL6, CLDN6, TBC1D2, GCG, NSUN7, C12orf37, KIAA2026, FANCC, RSPO2, CCR7, SCCPDH, PSG7, C14orf142, IQCF3, CN5H6.4, LPAR3, ST14, ST8SIA1, FAM65C, CXXC4, ASGR1, ZAN, C1orf64, CNTN5, OPALIN, RASGEF1B, LOC100240726, SPATA2, SLC36A4, FBXL17, SVEP1, ATP9A, PRAM1, OGFOD1, CEACAM21, CIT, BMX, TAGAP, C10orf35, SH3BP4, LOC339751, IL7R, MAPKAP1, KRT7, SAMD14, SPAG1, GPRIN3, CHDH, CARNS1, DENND1B, FHAD1, BIN2, LOC100293679, KCNA2, ZNF81, TRA2A, NCRNA00257, TMEM144, MS4A12, KL, ITGA9, TNFSF13, GPCPD1, NCRNA00221, NUDT22, CHGB, LOC440602, SDSL, ID2, PARD3B, LOC728705, SLC9A5, PTGDR, SARDH, CD1D, GRAMD4, LTB, RGS6, ERN2, CNR1, CRNDE, HIVEP3, TGM4, ALDOB, TPMT, SLC27A2, PTPRD, CASZ1, CAPN9, ZC3H12B, LOC646482, SPIRE2, SCML4, RNF213, PRSS8, STRBP, KLHL23, OR10H3, C14orf162, MRPS12, ITGB3, ITGB1BP1, WDHD1, C22orf45, FAM164A, IL4, SYDE1, NUSAP1, TAS2R4, ZNF451, C17orf99, C18orf62, VDR, PRR15, PGP, ATP6V0A2, DHDH, TRAF3IP2, MAST3, ERLIN1, C6orf142, TNXB, TRIM58, KIAA1543, SLC22A5, LOC151121, LOC553103, KIF23, TNFSF14, IL18R1, PDE4B, GKAP1, LOC285045, AKR1C3, VPS45, VWF, GH1, CCDC129, STEAP3, TSPAN17, PM20D2, PKHD1L1, ZNF608, SOX1, HMGB3, MYOZ2, FLJ37453, ATOH8, ERGIC1, SERPINA1, ATP8B1, KRT222, IL1B, FAM195A, VRK3, MRE11A, C18orf16, CNTNAP2, NEK11, EDN3, HNF4A, GLTSCR2, ARID5B, DAB2IP, TRIM50, PNPLA3, LOC389023, BCL2L2, DKFZp566F0947, MAOA, H2AFY2, HRK, SYNPR, BSND, GPD1, ALX4, CRMP1, SLC25A37, PRY, PTK2B, LOC340017, CAMK4, FANCI, C15orf48, ATP9B, ADAM3A, HOXA3, GNA14, MPP7, MBNL1, FLJ90680, C11orf93, SLC34A1, TMEM185A, C6orf182, KRT38, PAPLN, SEMA6A, HS6ST3, C5orf56, BRSK1, APCDD1, SLC35F1, CCDC125, FDX1, RFFL, SLC25A35, ANP32C, LOC100131733, APOA2, ID1, SV2B, GALNT2, LOC652993, ZNF552, TRAC, TOP2A, LYPD6, IQGAP3, ZNF627, CDH23, FNBP1L, PDE1B, LOC645638, NKD1, PPPIR9A, PSPH, GABRA1, C9orf68, GLTPD2, SAMD11, SLC35D1, CEACAM7, MAPK13, GAS2L1, ASF1B, TCF21, APBB1IP, CXCL14, CCDC67, HESS, BLM, TMEM54, STOX1, XYLT1, CELSR2, CDK1, CXCR6, SEMA3C, ZNF124, TIAL1, RREB1, APOB48R, HSD11B2, CENPA, LOC100289219, AQP11, GRAP2, CDC25A, FAM84A, PRLR, BTNL8, WNT5A, CEBPA, NCRNA00265, ARHGEF19, SOX6, PTTG1, ACVR1B, SAE1, MAEA, PPIF, ALDH2, AGMAT, FAM46C, PAPOLB or ASRGL1.

The TGF-β response signature of Endothelial Cells (E-TBRS) is selected from ASAP1, SAV1, WWTR1, ZNF532, ARL4C, PALM2-AKAP2, NRP1, CEP170, BNIP3L, RBMS1, MYOF, TGFB1, HIP1, ANGPT2, CDH2, VEGFA, UGCG, PLOD2, IDS, RNF13, SPOCK1, WWC2, FAP, EMP1, COL5A2, ALCAM, VCAN, CALD1, AKAP13, CHMP2B, STX7, MAP1B, DCBLD2, PTRF, FBN1, LRRFIP1, SLC25A36, SULF1, SEPT10, ACVR1, PAPPA, GNB5, CAMSAP1L1, QKI, PALLD, TNFRSF21, NRP2, UBE2H, FERMT2, CDC42BPA, ELK3, CALU, OSBPL1A, CLIC4, SMAD3, PICALM, PLAU, FN1, RHOQ, LRP12, FGFR1, BAG2, CCDC88A, KLF7, ANXA3, PTPRR, RDX, MPDZ, TRAM1, COL4A2, STAG2, TPM4, BGN, NOTCH2, BMPR2, TM6SF1, RRAS2, MEF2A, ZNF281, CALCRL, UBE2W, PTPN14, DPYSL3, IGFBP5, GPR176, FADS3, TRIB2, COL8A1, ODZ3, MACF1, TMEFF1, AKT3, MBNL2, LAMA4, TRAK1, ABCA1, EMCN, AIDA, FAM129A, THBS1, PIK3CA, SERPINB2, PERP, MCTP1, PDP1, OSMR, POLR2K, MAP1LC3B, EZR, ADAM10, ARPP19, LTBP1, CLDND1, DUSP3, TXNIP, SUSD5, ZEB1, BACH1, AZIN1, MAP4K5, DGKA, LEPR, PRKCH, PLEC, TCF7L2, BCAT1, LBH, PEA15, SLC39A6, DNAJB4, APLP2, PDLIM5, SAMD9, DEGS1, STC2, MLLT10, NMD3, SLC29A1, ATP6V1C1, TLK1, PPAP2B, FXR1, TGFB2, RANBP9, CHMP1B, ATP6V1G1, MCL1, SPTBN1, NCOA3, SMARCA2, MCFD2, TRIM23, NAB1, MAP3K7, TPP1, FZD6, MMP14, KIDINS220, HMOX1, SPSB1, BICD2, ZFHX3, OSBPL8, WSB1, APPL2, USO1, DDA1, PRKAR1A, IL6ST, IFNGR1, SNX3, TAB2, YWHAZ, SLC6A8, APBB2, MAF, CCNG2, SAR1A, RC3H2, CCL2, PDLIM7, HPS5, SLC7A11, ICAM1, FHL1, IFI16, VAMP3, FAM198B, F2R, RGS4, DNASE2, JAG1, IL32, SRGN, NCK1, DKK1, BEX4, TES, MYO6, ANTXR1, NUPR1, TUG1, TCF4, RASA3, ZMYM6, SS18, RAB23, CELF2, PRKCI, B3GNT2, KIAA1033, ARF6, GOLGA2, ANKRD12, PARVA, NID1, CLIP1, SEPT11, EID1, MET, PTPN12, SCARB2, C3orf52, PROSC, HSP90AA1, ITGA2, HIPK3, AOX1, TJP1, PRNP, LAPTM5, ARL6IP5, NA, SQLE, SEC23A, FKBP1A, SOAT1, TNFSF18, NPTX1, RAB3B, BCL2L1, CREG1, ITGB3, RIOK3, PKN2, ST6GALNAC4, IQGAP1, TRIO, TPR, DAB2, ITGB4, GNS, PPP1R2, LIG4, LYPLA1, PAK2, CLDN1, RNF146, EIF5A, PTK2, NBN, CPNE3, RAB14, PANX1, ARNTL, TWSG1, TSPAN3, IGF2BP3, PVR, YY1, PHTF2, CDK17, SLC6A15, HIST1H2BE, TBL1XR1, BCAP29, CSNK1A1, HDGFRP3, CBS, CD58, GLUD1, SLC16A1, SPAG9, PPFIBP1, CREBL2, PSEN1, RYK, F2RL1, MAX, ARF4, KIAA1109, B4GALT1, ROCK1, HSPG2, TANK, RAC2, PXN, TM9SF1, ECE1, IL8, DSTYK, SCAMP1, PLD1, LUC7L3, TPM1, PLAA, ARFGEF1, KDELR1, CCPG1, LAPTM4B, GLUD2, SERPINB9, PTP4A1, ATP2B1, HSPA13, WDFY3, MDFIC, TMCO3, IFI27, POLG, RALB, MTUS1, SOS2, OGT, ITGA6, GNB1, CBFB, SPIN1, LMAN1, PPPDE1, UBA6, ATP2B4, CDC73, WNK1, ZNF22, LPP, FLI1, NEDD4, PCDHGA3, PPP1R12A, PON2, UBE2D3, TTC3, MEIS3P1, CSDA, TMOD3, PTPN11, SH3BGRL, EIF3A, PHACTR2, CRK, UBE2J1, RYBP, DOCK9, SDPR, NCKAP1, CLDN11, NFAT5, SHC1, UBE2G1, RPRD1A, PPFIA1, EPAS1, NFATC2IP, EPB41L5, FGF2, KLHL7, SGPP1, HERC4, MICAL2, SP110, GSN, CCNA1, SEC14L1, RAB5C, RAD21, PTGS2, MAP3K2, UTP3, GLIPR1, NAA35, NBR1, TAF9B, NEDD9, TOX4, FLRT2, MBNL1, LOXL2, EPB41L3, NFIB, LIMS1, CALM1, TMX1, SIRPA, DCTD, CHP, NF1, PAFAH1B1, ACBD3, TFDP1, FEM1B, HHLA3, SYNM, YIPF5, NFYA, MGEA5, LYN, KITLG, CEACAM1, SNAP23, TPP2, NRAS, ASF1A, XPO7, CDC5L, RASA1, MARCH7, IL13RA1, ITPR3, GLG1, KPNA1, ATP13A3, EXOC5, LIPG, ABCF2, IL1RL1, ASNS, HSP90B1, ATG5, ELL2, WAC, ATP6AP2, FAIM, ABI1, ADH5, PDE4B, PWP1, ASPH, DNAJC10, SELT, SEPT2, GLUL, ENO1, PTPRF, CAPN7, RBM9, C5orf28, EIF4G1, HSF1, HSPH1, NONO, IKBKB, TNFRSF10C, TMEM41B, PTPRO, KPNA4, SPTLC1, NRCAM, RAB5A, TCF25, NUCB1, RCN2, ZNF148, LAMP1, SMC3, SOS1, GTF2I, RNFT1, IL1A, ITGA4, SLC16A3, RBM25, USP34, ANXA4, UBXN4, ETS1, DIAPH1, SYPL1, EXTL3, CALR, MGAT2, CREB1, G3BP2, SLC4A7, KLHL9, ELF1, YWHAB, CCDC6, SELE, MFSD6, SORBS3, CYP51A1, CD44, RAPGEF2, PIGC, EDEM3, ATP6V1A, ZFR, USP10, CPD, WTAP, GDE1, DHX9, UTRN, CDC27, GFRA1, TWF1, STIP1, PSAT1, GMFB, TNFRSF10D, RERE, COMT, NUP50, CD164, TGOLN2, WIZ, ENC1, M6PR, S100P, PGRMC1, RAD23A, NUDT4, LIN7C, GPD2, RABEP1, VDAC1, MED20, DMD, EML4, SPRY2, SLC11A2, SLC33A1, SMAD5, BCLAF1, BUB3, NUS1P3, SENP3, ZWILCH, PSG6, MAPK1, MANEA, SPAST, SNRNP27, RNF115, PSPH, TOR1AIP1, MED6, EPRS, VPS41, USP46, SMEK1, YWHAE, SCYL2, CTH, HIPK2, ACLY, TNFRSF10B, PRKACB, GBX2, MT1E, FBXW11, MT1X, DDX18, TRRAP, CLPTM1, GRB2, PAPOLA, PREPL, FNDC3A, MT1M, NFYB, POT1, PRPF4, GGA3, SNAPC3, SRSF2IP, ABCB1, CFLAR, KIAA0494, C1orf144, PTMS, KPNA6, C6orf106, RPE, MAGT1, SLC26A2, ABCC1, TPD52, LRP8, SYNCRIP, PCBD1, MAPKAPK2, SLC1A4, SMARCC1, SLC7A1, METAP2, ANKFY1, CLCN3, SNX4, MBTPS1, ALG13, GPR56, FOXJ3, MFNG, AP1S1, DCTN4, TSN, TRA2B, RIPK1, SERINC3, GM2A, TAGLN2, SMG1, OSBP, QPCT, MAN1A1, SH3GLB2, FUCA1, APBA1, GSPT1, MCM4, H2AFY, RANBP2, HADHA, TLR4, DAPK3, ELP3, SETD8, ARFIP1, ZFY, C19orf6, TGFBR2, BSG, NETO2, ENG, MAPK14, ADD1, FAS, CD46, ARF3, HMGA1, AGFG1, HNRNPC, MYO9B, YAP1, DDX3X, PCK2, PRPF40A, SEL1L, RAB6A, NOLC1, MAPRE1, CDV3, SKAP2, ACTR2, ZC3H15, ZMYND11, ITPR2, DAZAP2, EDC3, PCYOX1, MAP2K2, TFG, KCTD20, MTMR1, CAPRIN1, PIP5K1A, HNRNPH1, C14orf101, TMED2, CLN5, GRLF1, MPZL1, ROD1, SH3BP4, AP2B1, PRKDC, SEC23IP, RRN3P1, NUMA1, GDI2, ME2, CYB5R4, DCLRE1C, NAA15, NNT, TRIM27, DLG1, IGF2R, GTF2F1, TUBGCP2, ERC1, COPA, DLAT, SCP2, MAGOH, SRP72, TLE4, MAT2A, UBE4B, FAM120A, SOCS2, CANX, SSR1, AASDHPPT, API5, SLC30A1, GGA2, VAC14, KPNA3, TTC37, SSB, C1orf103, ARF1, MARCH6, RELN, ARHGDIA, C20orf30, PNO1, ADD3, PSME4, TFRC, PRPF4B, RAB1A, RHOBTB3, ZNF192, CHD4, ATP1B1, EIF1AX, DEDD, MOBKL1B, TP53, TMBIM6, HPCAL1, ANAPC5, STX16, EIF4E, MBTPS2, COMMD10, ERLIN1, SRSF10, PSG3, FKBP15, GLYR1, STS, TIA1, ANP32A, SYNJ1, KCTD5, NFYC, LARP4B, KDM6A, POLR2E, WDR1, CCT2, STX3, AMD1, PDE8A, U2AF2, BRCC3, SERBP1, KIF5B, HSPD1, POLDIP3, YKT6, UBE3B, SEC63, G3BP1, ILF3, CYB5B, ROCK2, PTBP1, KIAA0776, AGA, GATC, GPR137, GFPT1, STAM2, CDYL, SMPD1, MARCKS, UBE2G2, EPB41L2, AKIRIN1, MCM3AP, GART, ESF1, CLINT1, SRPR, SEL1L3, SLC35A2, SDHC, MMP1, AGPS, PLXNA2, HNRNPR, GRAP, SRRT, KIAA0182, C6orf62, ORC5L, NUDT21, LSS, CTSS, PDCD4, TBX1, RNF14, DIMT1L, PLEKHB2, SRSF1, CSTF2T, HS2ST1, CSNK2A1, TOP1, PTPRB, LSM12, USP1, ZFPL1, TSPAN12, SLC35A3, UBE2K, CCND2, DSCR3, GCLM, PAPSS2, SEMA3C, SRPK1, GOT1, ACOT7, DCAF7, IDH3A, GALNT2, PRPF6, YBX1, UBE2N, PPIF, HSPA9, LARP4, CYP20A1, SLC35D1, EPHB2 or TFAM.

The gene expression levels of TGF-β response signature in fibroblasts is determined by a Stromal Score or RNA sequence analysis.

### Stromal Score

The Stromal Score is a value between −1 through 1 which uses normalized RNA sequence data by a standardized Z-score. To determine and standardize the stromal score, the cancer tissue RNAseq data of TCGA (The Cancer Genome Altas, N>8000) pan-cancer database is analyzed. The median or mean value for the standard is set as the value of “0”. Using that standard as well as trial data, the range of RNAseq data values that equate the range between 0-1 and −1-0 is determined A high TGF-β response signature level is indicated as the value between 0 and 1, and a low TGF-β response signature level is indicated as the value between −1 and 0.

### RNA Sequence Analysis

The gene expression levels of TGF-β response signature in cells that comprise the tumor microenvironment (TME), which includes but is not limited to fibroblasts, T-cells, macrophages and endothelial cells can also be determined by RNA sequence analysis obtained from the patient. RNA sequence analysis examines the changing cellular transcriptome in total RNA, mRNA and miRNA by analyzing alternative gene spliced transcripts, post-transcriptional modifications, gene fusions, mutations/single-nucleotide polymorphisms (SNPs) and changes in gene expression. The types of analysis that are included in RNA sequence analysis include gene expression analysis, gene expression level correlation analysis, differentially expressed gene analysis, SNP analysis, gene ontology enrichment analysis and gene structure profiling.

### TGF-β Inhibitor

As used herein TGF-β inhibitor refers to a compound or an antibody which selectively inhibits the TGF-β type 1, 2 or 3 receptor, TGF-β ligand, or TGF-β mRNA expression. Exemplary inhibitors of TGF-β include, but are not limited to, Galunisertib (Eli Lilly), LY3200882 (Eli Lilly), NIS-793 (Novartis), SAR439459 (Sanofi) and M7824 (Merck Serono).

The contents of U.S. Pat. No. 8,080,568 filed Jun. 29, 2010 is incorporated herein by reference. In an embodiment of the present invention, the method of treating cancer patients relates to administering the compound represented by Formula 1, a pharmaceutically acceptable salt, solvate, or stereoisomer thereof, or a combination thereof disclosed in U.S. Pat. No. '568:

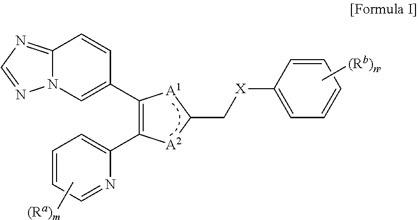

wherein, in Formula I, R^{a} may be each independently hydrogen (H), halo, C₁₋₆ alkyl, C₁₋₆ haloalkyl, C₃₋₆ cycloalkyl, OH, —O—C₁₋₆ alkyl, —O—C₁₋₆ haloalkyl, —O—C₃₋₆ cycloalkyl, —NH₂, —NH—C₁₋₆ alkyl, —NH—C₁₋₆ haloalkyl, —NH—C₃₋₆ cycloalkyl, —S—C₁₋₆ alkyl, —S—C₁₋₆ haloalkyl, —S—C₃₋₆ cycloalkyl, —CN, or —NO₂;

m may be 0, 1, 2, 3, or 4;

any one of A¹ and A² may be N, and the other is NR¹, wherein R¹ may be H, OH, C₁₋₆ alkyl, C₁₋₆ haloalkyl, or C₃₋₆ cycloalkyl;

X may be a bond, —(CH₂)ₚ—, —NR²—, —O—, or —S—, wherein p may be 0 or 1, and R² may be H or C₁₋₃ alkyl;

R^{b} may be each independently H, halo, C₁₋₆ alkyl, C₁₋₆ haloalkyl, C₃₋₆ cycloalkyl, C₂₋₆ alkenyl, C₂₋₆ alkynyl, —(CH₂)_{q}—OR³, —(CH₂)_{q}—NR³R⁴, —(CH₂)_{q}—SR³, —(CH₂)_{q}—NO₂, —(CH₂)_{q}—CONHOH, —(CH₂)_{q}—CN, —(CH₂)_{q}—COR³, —(CH₂)_{q}—CO₂R³, —(CH₂)_{q}—CONR³R⁴, —(CH₂)_{q}-tetrazole, —(CH₂)_{q}—CH═CH—CN, —(CH₂)_{q}—CH═CH—CO₂R³, —(CH₂)_{q}—CH═CH—CONR³R⁴, —(CH₂)_{q}—CH═CH-tetrazole, —(CH₂)_{q}—NHCOR³, —(CH₂)_{q}—NHCO₂R³, —(CH₂)_{q}—CONHSO₂R³, —(CH₂)_{q}—NHSO₂R³, —(CH₂)_{q}—C═C—CN, —(CH₂)_{q}—C═C—CO₂R³, —(CH₂)_{q}—C═C—CONR³R⁴, —(CH₂)_{q}—C═C-tetrazole, —(CH₂)_{q}—SOR⁵, —(CH₂)_{q}—SO₂R⁵, or —(CH₂)ᵣ—(OR³)₂,

wherein R³ and R⁴ may be each independently H, C₁₋₆ alkyl, C₁₋₆ haloalkyl, or C₃₋₆ cycloalkyl, or taken together with a nitrogen atom bound thereto to form a mono-cyclic ring, for example, imidazole, pyrrolidine, piperidine, morpholine, piperazine, and homopiperazine,

R⁵ may be C₁₋₆ alkyl, C₁₋₆ haloalkyl, or C₃₋₆ cycloalkyl,

q may be 0, 1, 2, 3, or 4,

r may be 1, 2, 3, or 4; and

n may be 0, 1, 2, 3, 4, or 5.

The alkyl group may be straight or branched. Examples of the alkyl group may include methyl, ethyl, propyl, isopropyl, butyl, isobutyl, sec-butyl, tert-butyl, n-pentyl, and n-hexyl. The alkyl group may be substituted with at least one selected from alkoxy, cycloalkoxy, amino, nitro, carboxy, cyano, halo, hydroxyl, sulfo, mercapto, or a combination thereof.

The cycloalkyl group may be cyclopropyl, cyclobutyl, cyclopentyl, or cyclohexyl.

The halo may be fluorine, chlorine, bromine, or iodine.

The alkenyl group may be straight or branched. The alkenyl group may be vinyl, allyl, isoprenyl, 2-butenyl, or 2-hexenyl. The alkenyl group may be substituted with alkoxy, cycloalkoxy, amino, nitro, carboxy, cyano, halo, hydroxyl, sulfo, mercapto, or a combination thereof.

The alkynyl group may be straight or branched. The alkynyl group may be ethynyl, propargyl, or 2-butynyl. The alkynyl group may be substituted with alkoxy, cycloalkoxy, amino, nitro, carboxy, cyano, halo, hydroxy, sulfo, mercapto, or a combination thereof.

In a preferred embodiment of the invention, the method of treating cancer patients relates to administering the compound represented by Formula II:

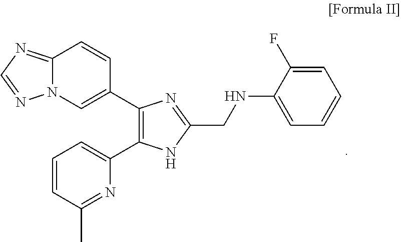 [SMILES: Cc1cccc(-c2[nH]c(CNc3ccccc3F)nc2-c2ccc3ncnn3c2)n1]

The compound of Formula II may be N-((4-([1,2,4]triazolo[1,5-a]pyridin-6-yl)-5-(6-methylpyridin-2-yl)-1H-imidazol-2-yl)methyl)-2-fluoroaniline or Vactosertib or TEW-7197.

The pharmaceutically acceptable salt may be a salt that may not cause significant irritation to an organism to which a compound is administered and may not abrogate the biological activity and properties of the compound. The salt may be, for example, an inorganic acid salt, an organic acid salt, or a metal salt. The inorganic acid salt may be a salt of hydrochloric acid, bromic acid, phosphoric acid, sulfuric acid, or disulfuric acid. The organic acid salt may be a salt of formic acid, acetic acid, propionic acid, lactic acid, oxalic acid, tartaric acid, malic acid, maleic acid, citric acid, fumaric acid, besylic acid, camsylic acid, edisylic acid, trichloroacetic acid, trifluoroacetic acid, benzoic acid, gluconic acid, methanesulfonic acid, glycolic acid, succinic acid, 4-toluenesulfonic acid, galacturonic acid, embonic acid, glutamic acid, ethanesulfonic acid, benzenesulfonic acid, p-toluenesulfonic acid, or aspartic acid. The metal salt may be a calcium salt, a sodium salt, a magnesium salt, a strontium salt, or a potassium salt.

The solvate may be a compound formed by attractive forces between solute and solvent molecules. The solvate may be a hydrate.

The stereoisomer refers to molecules that have the same molecular formula and connectivity of their atoms, but differ in spatial arrangement of atoms. The stereoisomer may be a diastereomer or an enantiomer of the compound of Formula I.

The human immunoglobulin herein refers to mixtures of antibodies. As used herein, the human immunoglobulin targets tumor cell evasion of immune checkpoints such as the cytotoxic T lymphocyte associated antigen-4 (CTLA-4), programmed cell death protein (PD-1) or the PD-1 ligand (PDL-1). Examples of the PD-L1 inhibitor may include BMS-936559 (MDX1105, Bristol Myers Squibb), MEDI4736 (MedImmune, AstraZeneca), MPDL3280A (Roche), and MSB0010718C (Merck). Examples of the PD1 inhibitor may include AMP-224 (Amplimmune, GlaxoSmith Klein), AMP-514 (MEDI0680, Amplimmune, GlaxoSmith Klein), nivolumab (Opdivo, Bristol Myers Squibb), Pembrolizumab (Keytruda, Merck), and Pidilizumab (Cure Tech). Examples of the CTLA4 inhibitor may include ipilimumab (Yervoy, Bristol Myers Squibb) and tremelimumab (Pfizer).

### Pharmaceutical Compositions

The term “pharmaceutical composition” is meant any composition, which contains at least one therapeutically or biologically active agent and is suitable for administration to the patient. Any of these formulations can be prepared by well-known and accepted methods of the art. See, for example, Remington: The Science and Practice of Pharmacy, 20th edition, (ed. A. R. Gennaro), Mack Publishing Co., Easton, Pa., 2000.

The administration of the TGF-β inhibitor compound, a pharmaceutically acceptable salt, solvate, stereoisomer thereof, or a combination thereof, and the human immunoglobulin may be performed directly by any suitable method, e.g., oral, intravenous, intramuscular, transdermal, mucosal, intranasal, intratracheal, or subcutaneous administration. The TGF-β inhibitor compound, a pharmaceutically acceptable salt, solvate, stereoisomer thereof, or a combination thereof, and the human immunoglobulin may be systemically or topically administered singly or together with another pharmaceutically active compound.

The TGF-β inhibitor compound, a pharmaceutically acceptable salt, solvate, stereoisomer thereof, or a combination thereof, and the human immunoglobulin may be administered simultaneously, individually, or sequentially.

When administrating the TGF-β inhibitor as a pharmaceutical for humans and other mammals, such as mice, rats, guinea-pigs, rabbits, cats, dogs, sheep, pigs, cattle, monkeys, baboons, chimpanzees, the TGF-β inhibitor is directly administered or are formulated into a dosage form using known pharmaceutical preparation methods. For example, according to the need, the drugs are taken orally, as sugar-coated tablets, capsules, elixirs and microcapsules, or non-orally, in the form of injections of sterile solutions or suspensions with water or any other pharmaceutically acceptable liquid. The compounds are mixed with pharmacologically acceptable carriers or medium, specifically, sterilized water, physiological saline, plant-oil, emulsifiers, suspending agents, surfactants, stabilizers, flavoring agents, excipients, vehicles, preservatives, binders and such, in a unit dose form required for generally accepted drug implementation. The amount of active ingredients in these preparations makes a suitable dosage within the indicated range acquirable.

Examples of additives that can be mixed to tablets and capsules are, binders such as gelatin, corn starch, tragacanth gum and arabic gum; excipients such as crystalline cellulose; swelling agents such as corn starch, gelatin and alginic acid; lubricants such as magnesium stearate; sweeteners such as sucrose, lactose or saccharin; flavoring agents such as peppermint, Gaultheria adenothrix oil and cherry. When the unit dosage form is a capsule, a liquid carrier, such as oil, can also be further included in the above ingredients. Sterile composites for injections can be formulated following normal drug implementations using vehicles such as distilled water used for injections.

Physiological saline, glucose, and other isotonic liquids including adjuvants, such as D-sorbitol, D-mannnose, D-mannitol, and sodium chloride, rare used as aqueous solutions for injections. These are used in conjunction with suitable solubilizers, such as alcohol, specifically ethanol, polyalcohols such as propylene glycol and polyethylene glycol, non-ionic surfactants, such as Polysorbate 80™ and HCO-50.

Sesame oil or Soy-bean oil is used as a oleaginous liquid and may be used in conjunction with benzyl benzoate or benzyl alcohol as a solubilizers and may be formulated with a buffer, such as phosphate buffer and sodium acetate buffer; a pain-killer, such as procaine hydrochloride; a stabilizer, such as benzyl alcohol, phenol; and an anti-oxidant. The prepared injection is filled into a suitable ampule.

Dosages for any one patient depends upon many factors, including the patient's size, body surface area, age, the particular nucleic acid to be administered, sex, time and route of administration, general health, and other drugs being administered concurrently.

In another embodiment, the present invention relates the method for selecting the cancer patient which is likely to benefit from adjuvant therapy comprising the step of determining the gene expression levels of TGF-β response signature, wherein increased gene expression levels of TGF-β response signature with respect to a reference value for the gene expression is indicative that the patient is likely to benefit from the therapy or wherein decreased gene expression levels of TGF-β response signature with respect to a reference value for the gene expression is indicative that the patient is unlikely to benefit from said therapy.

The invention also encompasses a kit for detecting the gene expression levels of TGF-β response signature in a biological sample. The kit contains a labeled compound for detecting TGF-β response signature protein or nucleic acid (e.g., mRNA or a monoclonal antibody) in a biological sample. Optionally, the kit contains a means for the gene expression levels of TGF-β response signature in the sample and means for comparing the gene expression levels of TGF-β response signature in the sample with a standard control value. The components of the kit are packaged together in a suitable container. The kit includes instructions for using the components to detect TGF-β response signature protein or nucleic acid.

The control sample is a value derived from a bodily tissue of an individual who is known not to have a cancer (e.g., a malignant tumor) based on earlier efforts to diagnose such pathologies. Alternatively, the control amount is an average of values from a plurality of non-cancerous individuals.

A purified or isolated polynucleotide (ribonucleic acid (RNA) or deoxyribonucleic acid (DNA)) or polypeptide is free of the genes or sequences or amino acids that flank it in its naturally-occurring state. An “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified also defines a degree of sterility that is safe for administration to a human subject, e.g., lacking infectious or toxic agents.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Complementary nucleic acid sequences hybridize under appropriate conditions to form stable duplexes containing few or no.

The terms “cell” and “cells”, which are meant to be inclusive, refer to one or more cells which can be in an isolated or cultured state, as in a cell line comprising a homogeneous or heterogeneous population of cells, or in a tissue sample, or as part of an organism, such as an insect larva or a transgenic mammal.

The term “amino acid” encompasses both naturally occurring and non-naturally occurring amino acids unless otherwise designated.

The term “an effective amount” means an amount of the substance in question which produces a statistically significant effect. For example, an “effective amount” for therapeutic uses is the amount of the composition comprising an active compound herein required to provide a clinically significant alteration in a measurable trait. For further example, by “an effective amount” is meant an amount of a compound, alone or in a combination, required to reduce or prevent the growth or invasiveness of a tumors of the reproductive system in a mammal. The effective amount of active compound(s) varies depending upon the route of administration, age, body weight, and general health of the subject. Such effective amounts will be determined using routine optimization techniques and are dependent on the particular condition to be treated, the condition of the patient, the route of administration, dosage required for the compounds of the invention is manifested as that which induces a statistically significant difference between treatment and control groups. Ultimately, the attending physician or veterinarian will decide the appropriate amount and dosage regimen.

A “therapeutically effective amount” refers to an amount effective, at dosages and for periods of time necessary, to achieve the desired therapeutic result. A therapeutically effective amount of modulator may vary according to factors such as the disease state, age, sex, and weight of the individual, and the ability of the modulator to elicit a desired response in the individual. Dosage regimens may be adjusted to provide the optimum therapeutic response. A therapeutically-effective amount is also one in which any toxic or detrimental effects of the modulator are outweighed by the therapeutically beneficial effects.

As used herein, the term “cell proliferative disorder” refers to conditions in which unregulated or abnormal growth, or both, of cells can lead to the development of an unwanted condition or disease, which may or may not be cancerous. A cell proliferative disorder includes a precancer or a precancerous condition. A cell proliferative disorder includes cancer. The methods and uses provided herein can be or may be used to treat or alleviate a symptom of cancer or to identify suitable candidates for such purposes. The term “cancer” includes solid tumors, as well as, hematologic tumors and/or malignancies. A “precancer cell” or “precancerous cell” is a cell manifesting a cell proliferative disorder that is a precancer or a precancerous condition. A “cancer cell” or “cancerous cell” is a cell manifesting a cell proliferative disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate molecular markers.

As used herein, “treating” or “treat” describes the management and care of a patient for the purpose of combating a disease, condition, or disorder and includes the administration of a compound of the present invention, or a pharmaceutically acceptable salt, prodrug, metabolite, polymorph or solvate thereof, to alleviate the symptoms or complications of a disease, condition or disorder, or to eliminate the disease, condition or disorder. The term “treat” can also include treatment of a cell in vitro or an animal model.

A compound of the present invention, or a pharmaceutically acceptable salt, prodrug, metabolite, polymorph or solvate thereof, can or may also be used to prevent a relevant disease, condition or disorder, or used to identify suitable candidates for such purposes. As used herein, “preventing,” “prevent,” or “protecting against” describes reducing or eliminating the onset of the symptoms or complications of such disease, condition or disorder.

As used herein, the term “alleviate” is meant to describe a process by which the severity of a sign or symptom of a disorder is decreased Importantly, a sign or symptom can be alleviated without being eliminated. The administration of pharmaceutical compositions of the invention can or may lead to the elimination of a sign or symptom, however, elimination is not required. Effective dosages should be expected to decrease the severity of a sign or symptom. For instance, a sign or symptom of a disorder such as cancer, which can occur in multiple locations, is alleviated if the severity of the cancer is decreased within at least one of multiple locations.

As used herein, a “subject” is interchangeable with a “subject in need thereof” or a “patient”, which refer to a subject having a cell proliferation disorder, or a subject having an increased risk of developing such disorder relative to the population at large. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In one embodiment, the mammal is a human A subject in need thereof can be one who has been previously diagnosed or identified as having cancer or a precancerous condition. A subject in need thereof can also be one who has (e.g., is suffering from) cancer or a precancerous condition. Alternatively, a subject in need thereof can be one who has an increased risk of developing such disorder relative to the population at large (i.e., a subject who is predisposed to developing such disorder relative to the population at large). A subject in need thereof can have a precancerous condition. The term “animal” includes human beings.

As used herein, a “responder” refers to a patient whose best overall response on treatment is stable disease (SD) or greater, and a “non-responder” refers to a patient whose best overall response on treatment is progression on disease (PD) or worse.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The present embodiments may have different forms and should not be construed as being limited to the descriptions set forth herein. Accordingly, the embodiments are merely described to explain aspects of the present description. As used herein, the term “and/or” includes any and all combinations of one or more of the associated listed items.

### Example 1

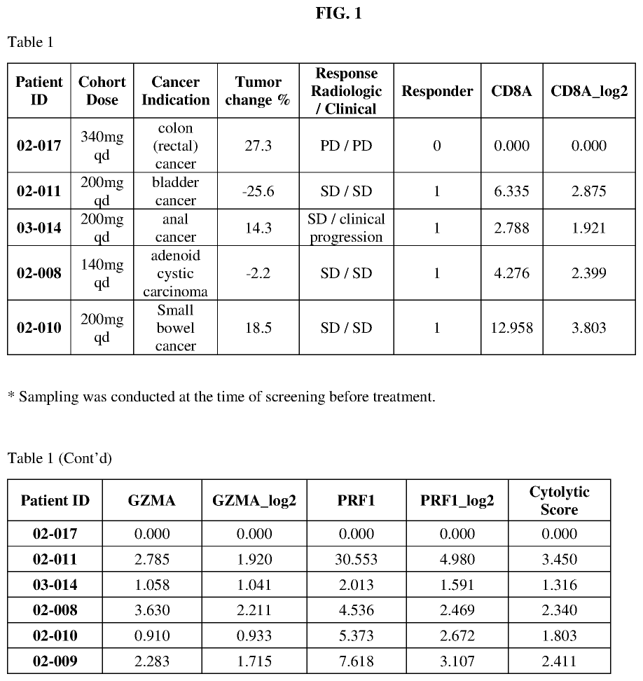

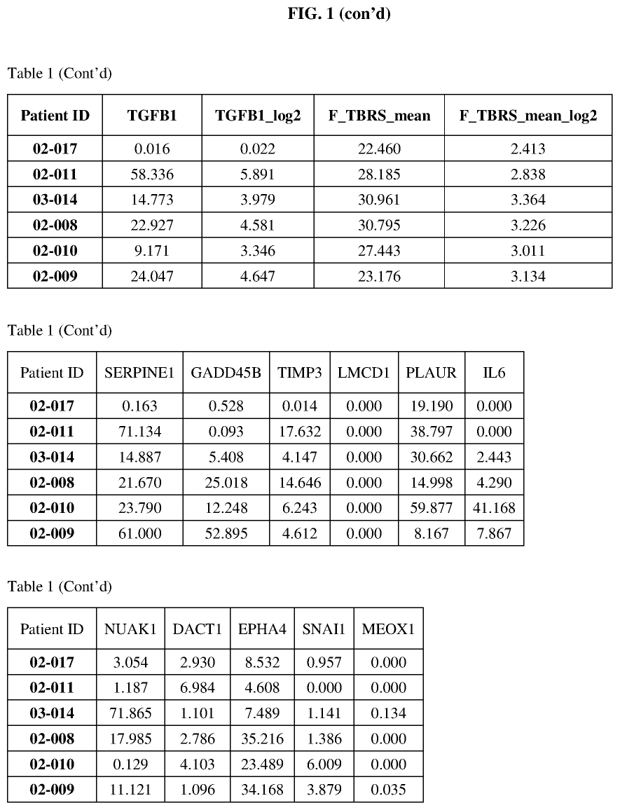

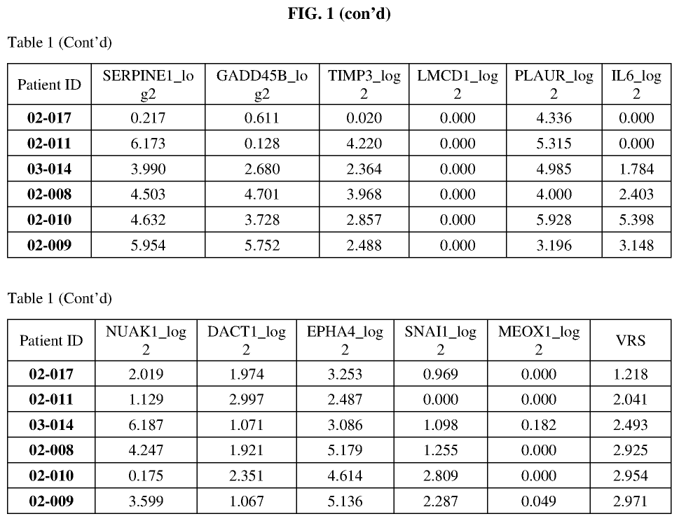

The RNAseq and tumor assessment data of the biopsy samples of patients with advanced solid tumors are shown in FIG. 1. For example, the F-TBRS data (“F_TBRS_mean”) and the cytolytic score (“CytScore”) data are shown in FIG. 1.

A ‘responder’ refers to a Patient whose best overall response on treatment was stable disease (SD) or greater, and a ‘non-responder’ refers to a patient whose best overall response on treatment was progression on disease (PD) or worse. The unit for all data of gene expression is TPM (Transcripts Per Kilobase Million). The CD8A gene is implicated in playing an important role in the immune response. The cytolytic score is used to assess anticancer immunity. The F-TBRS comprises the genes FLT1, COL10A1, IGFBP3, NOX4, MEX3B, GAS1, INHBA, VEGFA, CDKN2B, NA, FBXO32, CALB2, CTGF, KANK4, NET1, HEY1, SERPINE1, ESM1, TIMP3, SYNE1, BHLHE40, PLAUR, APBB2, FGF1, ANGPTL4, LMCD1, PGM2L1, KAL1, TNFAIP6, OSGIN2, PODXL, PCDH9, C13orf33, RASGRP3, LOH3CR2A, SPSB1, FN1, GADD45B, TRIB1, STK17B, KLF7, LRRC8C, FNIP2, TGFB2, FRMD4A, CNTN1, NGF, NUAK1, EFNB2, TSPAN2, CHST11, EGR2, DAAM1, PALLD, GPR161, CALD1, LOC728449, MGC16121, HIVEP2, RHOU, KDM6B, FOXP1, ELMOD1, SEMA7A, PTHLH, PMEPA1, HAS2, SNORD114-3, GRB14, LIF, TSHZ3, PDLIM4, LOC728264, ZNF365, PDGFC, JUNB, CILP, BPGM, ARHGEF3, PGBD5, TAGLN3, TUFT1, GPR183, S1PR5, CLDN4, MBOAT2, CNNM4, DNAJB5, C3orf52, DHRS2, SOX4, EPHA4, COL27A1, SMAD7, F2RL1, LOC100128178, RASL12, SLC35F2, SETBP1, DOCK10, C5orf13, DNAJC18, DACT1, WNT9A, ETV6, FGF18, HBEGF, TNC, SDC1, KIAA1755, EDN1, ITGB6, MURC, PTGS2, PLEK2, LOC201651, STEAP2, SLC46A3, SNX30, RYBP, TMEM49, SORBS2, HIC1, NEDD9, ARHGEF40, IFIH1, GZMK, VEPH1, PIK3CD, IL6, YIPF5, SKIL, RASD1, JARID2, IL11, SNAI1, SOX6, STK38L, NKX3-1, CDH6, PELI1, PRDM1, PDPN, WNT2, LMO4, C4orf26, CACHD1, PRR5L, TMEM2, DDX10, MTSS1, CLDN14, JHDM1D, SLC19A2, PLCE1, PRR9, MEGF9, GOPC, MSC, PPP1R14C, PKNOX2, MSX2, SNCAIP, SLC35F3, LOC727930, HS3ST3B1, MEOX1, E2F7, AUTS2, FUT4, DLX2 or TBX3. A subset of 11 genes comprising SERPINE1(PAI1), GADD45B, TIMP3, LMCD1, PLAUR, IL6, NUAK1, DACT1, EPHA4, SNAI1, and MEOX1 is provided as a new TGF-β response signature.

### Example 2

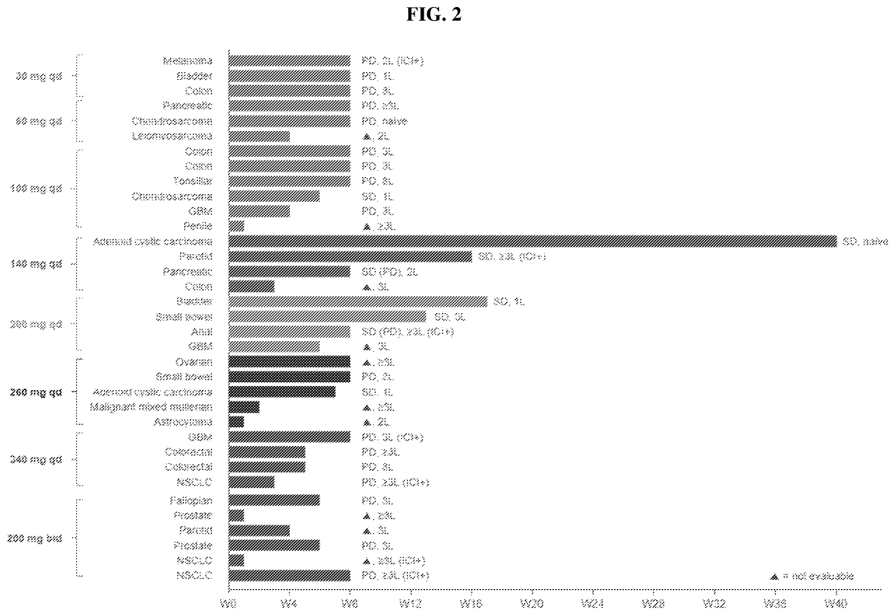

Exemplary anti-tumor activity of Vactosertib (the compound represented by Formula II; TEW-7197) is shown in FIG. 2. At higher doses (e.g., ≥140 mg, qd), seven patients achieved SD (stable disease, responder). In FIG. 2, “qd” refers to quaque die (daily dosing); ‘bid” refers to bis in die (twice daily dosing); “ICI+” refers to immune checkpoint inhibitor treated; and “nL” refers to the number of lines of prior treatment. For example, “2L” refers to two prior lines of treatment. The symbol triangle (▴) denotes “not evaluable”.

### Example 3

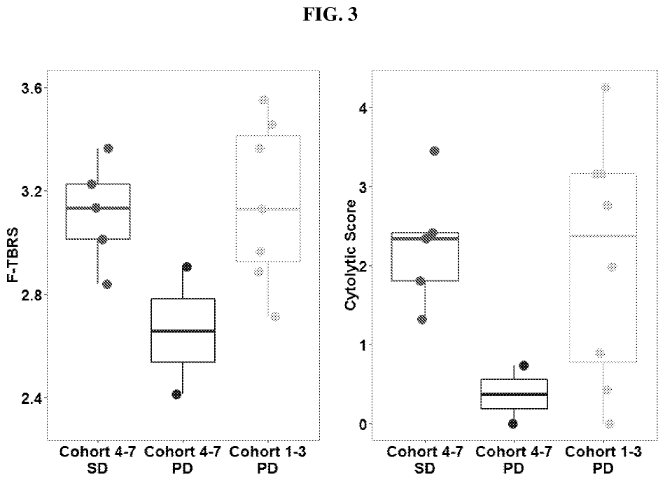

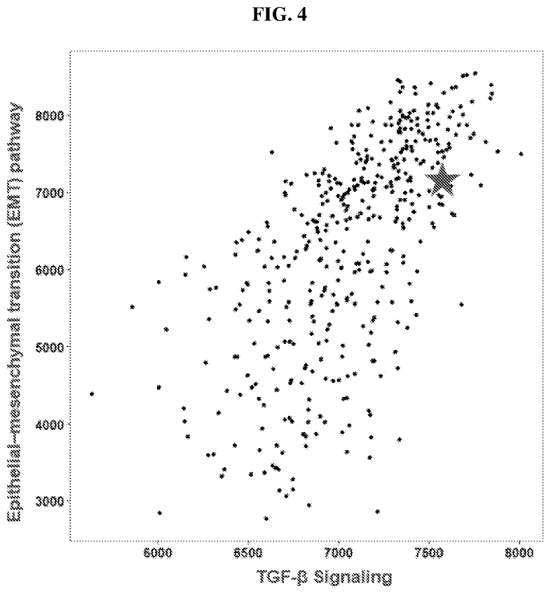

FIG. 3 and FIG. 4 demonstrate that high TBRS predicts responders of Vactosertib (the compound represented by Formula II; TEW-7197). In particular, the F-TBRS and the cytolytic score in cancer of responders and non-responders at the Phase I trial of single administration of Vactosertib (the compound represented by Formula II; TEW-7197) were analyzed and the results were shown in FIG. 3. At the Phase I trial, responders (SD patients) in cohort 4-7 (≥140 mg) showed high F-TBRS and high cytolytic score (GZMA+PRF1).

In addition, the EMT/F-TBRS expression distribution from the TCGA database of bladder cancer was analyzed and the results are shown in FIG. 4. The patient with bladder cancer who showed −25% tumor shrinkage represents the patient subgroup with EMT signature and enhanced TGF-β activity among patients with bladder cancer in the TCGA database. The star (★) represents the expression level of EMT/F-TBRS in the cancer from a patient of which bladder cancer was reduced by approximately 25% at the Phase I trial.

These results clearly indicate that the patients with bladder cancer in which the expression level of EMT/F-TBRS is high may obtain significantly superior effects by the treatment with Vactosertib or the combinational treatment of Vactosertib and immune-oncology therapy.

### List of Genes

The access number of the genes described in the application is listed in the following table:

| Gene | Accession No. | Gene | Accession No. | Gene | Accession No. |
|---|---|---|---|---|---|
| FLT1 | ENSG00000102755 | LOC401097 (or | ENSG00000180044 | SPOCK1 | ENSG00000152377 |
| | | C3orf80) | | | |
| COL10A1 | ENSG00000123500 | C17orf91 | ENSG00000186594 | WWC2 | ENSG00000151718 |
| IGFBP3 | EN5G00000146674 | NLRP12 | EN5G00000142405 | FAP | EN5G00000078098 |
| NOX4 | EN5G00000086991 | USP53 | EN5G00000145390 | EMP1 | EN5G00000134531 |
| MEX3B | ENSG00000183496 | C19orf59 | ENSG00000183019 | COL5A2 | ENSG00000204262 |
| GAS1 | EN5G00000180447 | NPR1 | EN5G00000169418 | ALCAM | EN5G00000170017 |
| INHBA | EN5G00000122641 | FMNL3 | EN5G00000161791 | AKAP13 | EN5G00000170776 |
| VEGFA | ENSG00000112715 | SLC41A1 | ENSG00000133065 | CHMP2B | ENSG00000083937 |
| CDKN2B | ENSG00000147883 | KCNA3 | ENSG00000177272 | STX7 | ENSG00000079950 |
| NA (or XK) | ENSG00000047597 | CACNA2D1 | ENSG00000153956 | MAP1B | ENSG00000131711 |
| FBXO32 | ENSG00000156804 | ARID3B | ENSG00000179361 | DCBLD2 | ENSG00000057019 |
| CALB2 | ENSG00000172137 | GAPT | ENSG00000175857 | PTRF | ENSG00000177469 |
| CTGF | ENSG00000118523 | BLOC1S2 | ENSG00000196072 | FBN1 | ENSG00000166147 |
| KANK4 | ENSG00000132854 | IGF2BP1 | ENSG00000159217 | LRRFIP1 | ENSG00000124831 |
| NET1 | ENSG00000173848 | PCNX | ENSG00000100731 | SLC25A36 | ENSG00000114120 |
| HEY1 | ENSG00000164683 | SCD | ENSG00000099194 | 10-Sep. | EN5G00000186522 |
| SERPINE1 | EN5G00000106366 | CCDC157 | EN5G00000187860 | ACVR1 | ENSG00000115170 |
| ESM1 | EN5G00000164283 | BLMH | EN5G00000108578 | PAPPA | EN5G00000182752 |
| TIMP3 | EN5G00000100234 | SLC5A7 | EN5G00000115665 | GNB5 | EN5G00000069966 |
| SYNE1 | ENSG00000131018 | MFI2 | EN5G00000163975 | CAMSAP1L1 | ENSG00000118200 |
| BHLHE40 | EN5G00000134107 | FKBP15 | EN5G00000119321 | QKI | EN5G00000112531 |
| PLAUR | EN5G00000011422 | TRMT1 | EN5G00000104907 | TNFRSF21 | EN5G00000146072 |
| APBB2 | EN5G00000163697 | FCGR3B | EN5G00000162747 | NRP2 | EN5G00000118257 |
| FGF1 | EN5G00000113578 | TEX14 | ENSG00000121101 | UBE2H | EN5G00000186591 |
| ANGPTL4 | EN5G00000167772 | OK/SW-CL.36 | EN5G00000172270 | FERMT2 | EN5G00000073712 |
| | | (or BSG basigin) | | | |
| LMCD1 | EN5G00000071282 | COX15 | EN5G00000014919 | CDC42BPA | EN5G00000143776 |
| PGM2L1 | EN5G00000165434 | MEX3D | EN5G00000181588 | CALU | EN5G00000128595 |
| KAL1 | ENSG00000011201 | LTA4H | ENSG00000111144 | OSBPL1A | EN5G00000141447 |
| TNFAIP6 | EN5G00000123610 | AKR1C1 | EN5G00000187134 | SMAD3 | EN5G00000166949 |
| OSGIN2 | EN5G00000164823 | CNIH4 | EN5G00000143771 | PICALM | EN5G00000073921 |
| PODXL | EN5G00000128567 | NPTXR | EN5G00000221890 | RHOQ | EN5G00000119729 |
| PCDH9 | EN5G00000184226 | TP53INP2 | EN5G00000078804 | LRP12 | EN5G00000147650 |
| C13orf33 | EN5G00000102802 | C12orf59 | EN5G00000165685 | FGFR1 | EN5G00000077782 |
| RASGRP3 | EN5G00000152689 | KLHL29 | EN5G00000119771 | BAG2 | EN5G00000112208 |
| LOH3CR2A (or | NC_000003.12 | LARGE (or | EN5G00000133424 | ANXA3 | EN5G00000138772 |
| LINC00312) | | LARGE1) | | | |
| SPSB1 | EN5G00000171621 | MMP2 | EN5G00000087245 | PTPRR | EN5G00000153233 |
| FN1 | EN5G00000115414 | KCNC3 | EN5G00000131398 | RDX | EN5G00000137710 |
| GADD45B | ENSG00000099860 | NAPSB | ENSG00000131401 | MPDZ | ENSG00000107186 |
| TRIB1 | EN5G00000173334 | ACADVL | EN5G00000072778 | TRAM1 | EN5G00000067167 |
| STK17B | ENSG00000081320 | C1orf126 | EN5G00000175147 | COL4A2 | EN5G00000134871 |
| KLF7 | ENSG00000118263 | CYP19A1 | ENSG00000137869 | STAG2 | ENSG00000101972 |
| LRRC8C | ENSG00000171488 | TNNT2 | ENSG00000118194 | TPM4 | ENSG00000167460 |
| FNIP2 | EN5G00000052795 | NRK | EN5G00000123572 | BGN | EN5G00000182492 |
| TGFB2 | EN5G00000092969 | MSTN | EN5G00000138379 | NOTCH2 | EN5G00000134250 |
| FRMD4A | ENSG00000151474 | CYLD | ENSG00000083799 | BMPR2 | ENSG00000204217 |
| CNTN1 | ENSG00000018236 | RGS8 | ENSG00000135824 | RRAS2 | ENSG00000133818 |
| NGF | ENSG00000134259 | HDGFRP3 | ENSG00000166503 | CALCRL | ENSG00000064989 |
| NUAK1 | ENSG00000074590 | HS3ST5 | ENSG00000249853 | UBE2W | ENSG00000104343 |
| EFNB2 | EN5G00000125266 | LOC255167 (or | ENSG00000250056 | PTPN14 | ENSG00000152104 |
| | | LINC01018) | | | |
| TSPAN2 | ENSG00000134198 | KCNIP2 | ENSG00000120049 | IGFBP5 | ENSG00000115461 |
| CHST11 | ENSG00000171310 | IL17RB | ENSG00000056736 | GPR176 | ENSG00000166073 |
| EGR2 | ENSG00000122877 | ANG | ENSG00000214274 | TRIB2 | ENSG00000071575 |
| DAAM1 | ENSG00000100592 | FU13197 (or | ENSG00000231160.9 | COL8A1 | ENSG00000144810 |
| | | KLF3-AS1) | | | |
| PALLD | ENSG00000129116 | TPSB2 | EN5G00000197253 | ODZ3 (or | EN5G00000218336 |
| | | | | TENM3) | |
| GPR161 | ENSG00000143147 | FURIN | ENSG00000140564 | MACF1 | ENSG00000127603 |
| CALD1 | ENSG00000122786 | NEK6 | ENSG00000119408 | TMEFF1 | ENSG00000241697 |
| LOC728449 (or | EN5G00000264230 | CD58 | EN5G00000116815 | AKT3 | EN5G00000117020 |
| ANXA8L1) | | | | | |
| MGC16121 (or | ENSG00000223749 | PYROXD1 | ENSG00000121350 | MBNL2 | ENSG00000139793 |
| MIR503HG) | | | | | |
| HIVEP2 | ENSG00000010818 | ALK | ENSG00000171094 | LAMA4 | ENSG00000112769 |
| RHOU | ENSG00000116574 | GOLT1A | ENSG00000174567 | TRAK1 | ENSG00000182606 |
| KDM6B | EN5G00000132510 | CNKSR3 | EN5G00000153721 | ABCA1 | EN5G00000165029 |
| FOXP1 | EN5G00000114861 | CHD9 | EN5G00000177200 | EMCN | EN5G00000164035 |
| ELMOD1 | ENSG00000110675 | RHOH | ENSG00000168421 | AIDA | ENSG00000186063 |
| SEMA7A | ENSG00000138623 | L3MBTL4 | ENSG00000154655 | FAM129A | ENSG00000135842 |
| PTHLH | ENSG00000087494 | HAL | ENSG00000084110 | PIK3CA | ENSG00000121879 |
| PMEPA1 | ENSG00000124225 | S100A8 | ENSG00000143546 | SERPINB2 | ENSG00000197632 |
| HAS2 | EN5G00000170961 | NLRC4 | EN5G00000091106 | PERP | EN5G00000112378 |
| SN0RD114-3 | ENSG00000201839 | PTGER3 | ENSG00000050628 | MCTP1 | ENSG00000175471 |
| GRB14 | ENSG00000115290 | CLP1 | ENSG00000172409 | PDP1 | ENSG00000164951 |
| LIF | ENSG00000128342 | SLC35F4 | ENSG00000151812 | OSMR | ENSG00000145623 |
| TSHZ3 | ENSG00000121297 | IL27RA | ENSG00000104998 | POLR2K | ENSG00000147669 |
| PDLIM4 | EN5G00000131435 | PARVA | EN5G00000197702 | MAP1LC3B | EN5G00000140941 |
| LOC728264 (or | EN5G00000249669 | CARD11 | EN5G00000198286 | EZR | EN5G00000092820 |
| CARMN) | | | | | |
| ZNF365 | ENSG00000138311 | MTMR11 | ENSG00000014914 | ADAM10 | ENSG00000137845 |
| PDGFC | ENSG00000145431 | FSTL5 | ENSG00000168843 | ARPP19 | ENSG00000128989 |
| JUNB | EN5G00000171223 | CSN1S1 | EN5G00000126545 | LTBP1 | EN5G00000049323 |
| CILP | ENSG00000138615 | ANK1 | ENSG00000029534 | CLDND1 | ENSG00000080822 |
| BPGM | EN5G00000172331 | BCAS4 | EN5G00000124243 | DUSP3 | EN5G00000108861 |
| ARHGEF3 | ENSG00000163947 | PPIH | ENSG00000171960 | TXNIP | ENSG00000265972 |
| PGBD5 | EN5G00000177614 | FOS | EN5G00000170345 | SUSD5 | EN5G00000173705 |
| TAGLN3 | ENSG00000144834 | MIA2 | ENSG00000150527 | BACH1 | ENSG00000156273 |
| TUFT1 | ENSG00000143367 | FOXQ1 | ENSG00000164379 | AZIN1 | ENSG00000155096 |
| GPR183 | ENSG00000169508 | MNDA | ENSG00000163563 | MAP4K5 | ENSG00000012983 |
| S1PR5 | ENSG00000180739 | SLC1A2 | ENSG00000110436 | DGKA | ENSG00000065357 |
| CLDN4 | ENSG00000189143 | K1AA1274 | ENSG00000107719 | LEPR | ENSG00000116678 |
| MBOAT2 | ENSG00000143797 | COQ2 | ENSG00000173085 | PRKCH | ENSG00000027075 |
| CNNM4 | ENSG00000158158 | WLS | ENSG00000116729 | PLEC | ENSG00000178209 |
| DNAJB5 | ENSG00000137094 | MSRA | ENSG00000175806 | TCF7L2 | ENSG00000148737 |
| C3orf52 | ENSG00000114529 | APOC2 | ENSG00000234906 | BCAT1 | ENSG00000060982 |
| DHRS2 | ENSG00000100867 | GSN | ENSG00000148180 | LBH | ENSG00000213626 |
| SOX4 | ENSG00000124766 | TFAP2B | ENSG00000008196 | PEA15 | ENSG00000162734 |
| EPHA4 | ENSG00000116106 | CCNA1 | ENSG00000133101 | SLC39A6 | ENSG00000141424 |
| COL27A1 | ENSG00000196739 | LRRC1 | ENSG00000137269 | DNAJB4 | ENSG00000162616 |
| SMAD7 | ENSG00000101665 | PRDM14 | ENSG00000147596 | APLP2 | ENSG00000084234 |
| F2RL1 | ENSG00000164251 | SMAD6 | ENSG00000137834 | PDLIM5 | ENSG00000163110 |
| LOC100128178 | ENSG00000230630 | ST8SIA2 | ENSG00000140557 | SAMD9 | ENSG00000205413 |
| (or DNM3OS) | | | | | |
| RASL12 | ENSG00000103710 | PHGDH | ENSG00000092621 | DEGS1 | ENSG00000143753 |
| SLC35F2 | ENSG00000110660 | TMEM111 | ENSG00000125037 | STC2 | ENSG00000113739 |
| SETBP1 | ENSG00000152217 | PSEN1 | ENSG00000080815 | MLLT10 | ENSG00000078403 |
| DOCK10 | ENSG00000135905 | HIPK2 | ENSG00000064393 | NMD3 | ENSG00000169251 |
| C5orf13 | EN5G00000134986 | ARPP21 | EN5G00000172995 | ATP6V1C1 | EN5G00000155097 |
| DNAJC18 | ENSG00000170464 | SBNO1 | ENSG00000139697 | TLK1 | ENSG00000198586 |
| DACT1 | ENSG00000165617 | SP100 | ENSG00000067066 | PPAP2B | ENSG00000162407 |
| WNT9A | ENSG00000143816 | PKD2L1 | ENSG00000107593 | FXR1 | ENSG00000114416 |
| ETV6 | EN5G00000139083 | FBP1 | EN5G00000165140 | RANBP9 | ENSG00000010017 |
| FGF18 | EN5G00000156427 | TTC12 | EN5G00000149292 | CHMP1B | EN5G00000255112 |
| HBEGF | ENSG00000113070 | FAM71F2 | EN5G00000205085 | ATP6V1G1 | EN5G00000136888 |
| TNC | EN5G00000041982 | TOPORS | EN5G00000197579 | MCL1 | EN5G00000143384 |
| SDC1 | EN5G00000115884 | B3GNT5 | EN5G00000176597 | NCOA3 | EN5G00000124151 |
| K1AA1755 | ENSG00000149633 | GPR25 | ENSG00000170128 | SMARCA2 | ENSG00000080503 |
| EDN1 | ENSG00000078401 | EWSR1 | EN5G00000182944 | MCFD2 | EN5G00000180398 |
| ITGB6 | ENSG00000115221 | ADC | EN5G00000142920 | TRIM23 | EN5G00000113595 |
| MURC | ENSG00000170681 | FBXO9 | ENSG00000112146 | NAB1 | ENSG00000138386 |
| PTGS2 | EN5G00000073756 | DYX1C1 | EN5G00000256061 | MAP3K7 | EN5G00000135341 |
| PLEK2 | ENSG00000100558 | CELF6 | ENSG00000140488 | TPP1 | ENSG00000166340 |
| LOC201651 (or | ENSG00000240602 | TMEM38B | ENSG00000095209 | FZD6 | ENSG00000164930 |
| AADACP1) | | | | | |
| STEAP2 | ENSG00000157214 | TMED5 | ENSG00000117500 | KIDINS220 | ENSG00000134313 |
| SLC46A3 | ENSG00000139508 | ZNF527 | ENSG00000189164 | HMOX1 | ENSG00000100292 |
| SNX30 | ENSG00000148158 | MEX3C | ENSG00000176624 | BICD2 | ENSG00000185963 |
| RYBP | ENSG00000163602 | LRRC27 | ENSG00000148814 | ZFHX3 | ENSG00000140836 |
| TMEM49 | ENSG00000062716 | GCHFR | ENSG00000137880 | OSBPL8 | ENSG00000091039 |
| SORBS2 | ENSG00000154556 | PGLYRP2 | ENSG00000161031 | WSB1 | ENSG00000109046 |
| HIC1 | ENSG00000177374 | LMX1A | ENSG00000162761 | APPL2 | ENSG00000136044 |
| NEDD9 | ENSG00000111859 | GPR34 | ENSG00000171659 | USO1 | ENSG00000138768 |
| ARHGEF40 | ENSG00000165801 | CADM4 | ENSG00000105767 | PRKAR1A | ENSG00000108946 |
| IFIH1 | ENSG00000115267 | RIT2 | ENSG00000152214 | IL6ST | ENSG00000134352 |
| GZMK | ENSG00000113088 | UTY | EN5G00000183878 | IFNGR1 | EN5G00000027697 |
| VEPH1 | ENSG00000197415 | ARFGEF2 | ENSG00000124198 | SNX3 | ENSG00000112335 |
| PIK3CD | ENSG00000171608 | NUP62CL | EN5G00000198088 | TAB2 | EN5G00000055208 |
| IL6 | ENSG00000136244 | PDCD1 | ENSG00000188389 | YWHAZ | ENSG00000164924 |
| YIPF5 | ENSG00000145817 | PTPDC1 | ENSG00000158079 | MAF | ENSG00000178573 |
| SKIL | ENSG00000136603 | PWRN2 | ENSG00000260551 | CCNG2 | ENSG00000138764 |
| RASD1 | ENSG00000108551 | PTPRZ1 | EN5G00000106278 | SAR1A | EN5G00000079332 |
| JARID2 | ENSG00000008083 | CDK3 | ENSG00000250506 | RC3H2 | ENSG00000056586 |
| IL11 | EN5G00000095752 | RBP4 | EN5G00000138207 | CCL2 | ENSG00000108691 |
| SNAI1 | ENSG00000124216 | PLAC1L | ENSG00000149507 | HPS5 | ENSG00000110756 |
| SOX6 | ENSG00000110693 | C12orf49 | ENSG00000111412 | SLC7A11 | ENSG00000151012 |
| STK38L | ENSG00000211455 | LOC339807 (or | ENSG00000233694 | ICAM1 | ENSG00000090339 |
| | | LINC02579) | | | |
| NKX3-1 | ENSG00000167034 | DYNC1H1 | ENSG00000197102 | FHL1 | ENSG00000022267 |
| CDH6 | ENSG00000113361 | LRRC6 | ENSG00000129295 | IFI16 | ENSG00000163565 |
| PELI1 | ENSG00000197329 | EFCAB5 | ENSG00000176927 | VAMP3 | ENSG00000049245 |
| PRDM1 | ENSG00000057657 | SLC46A2 | ENSG00000119457 | FAM198B | ENSG00000164125 |
| PDPN | ENSG00000162493 | NCR3 | EN5G00000204475 | F2R | ENSG00000181104 |
| WNT2 | ENSG00000105989 | TCEB3B | ENSG00000206181 | RGS4 | ENSG00000117152 |
| LMO4 | ENSG00000143013 | TUBE1 | EN5G00000074935 | IL32 | ENSG00000008517 |
| C4orf26 | ENSG00000174792 | FCRL1 | ENSG00000163534 | SRGN | ENSG00000122862 |
| CACHD1 | EN5G00000158966 | GPR37 | EN5G00000170775 | NCK1 | EN5G00000158092 |
| PRR5L | EN5G00000135362 | RALA | ENSG00000006451 | BEX4 | ENSG00000102409 |
| TMEM2 | ENSG00000135048 | SYTL1 | ENSG00000142765 | ANTXR1 | ENSG00000169604 |
| DDX10 | ENSG00000178105 | NKTR | ENSG00000114857 | TUG1 | ENSG00000253352 |
| MTSS1 | ENSG00000170873 | CES4 | ENSG00000228695 | TCF4 | ENSG00000196628 |
| CLDN14 | EN5G00000159261 | CAMP (or | EN5G00000108946 | RASA3 | EN5G00000185989 |
| | | PRKAR1A) | | | |
| JHDM1D | ENSG00000006459 | DDX52 | ENSG00000278053 | ZMYM6 | ENSG00000163867 |
| SLC19A2 | ENSG00000117479 | EPB41L5 | ENSG00000115109 | CELF2 | ENSG00000048740 |
| PLCE1 | ENSG00000138193 | FOXG1 | ENSG00000176165 | PRKCI | ENSG00000163558 |
| PRR9 | ENSG00000203783 | GOLGA8IP | ENSG00000277561 | B3GNT2 | ENSG00000170340 |
| MEGF9 | ENSG00000106780 | FLJ33996 (or | ENSG00000196876 | KIAA1033 | ENSG00000136051 |
| | | SCN8A) | | | |
| GOPC | ENSG00000047932 | GGT7 | ENSG00000131067 | ARF6 | ENSG00000165527 |
| MSC | ENSG00000178860 | SLC35D2 | ENSG00000130958 | GOLGA2 | ENSG00000167110 |
| PPP1R14C | ENSG00000198729 | TCTN2 | ENSG00000168778 | ANKRD12 | ENSG00000101745 |
| PKNOX2 | ENSG00000165495 | MYO6 | ENSG00000196586 | NID1 | ENSG00000116962 |
| MSX2 | ENSG00000120149 | SLC44A5 | ENSG00000137968 | CLIP1 | ENSG00000130779 |
| SNCAIP | ENSG00000064692 | ATP13A3 | ENSG00000133657 | 11-Sep. | EN5G00000138758 |
| SLC35F3 | EN5G00000183780 | LIPG | ENSG00000101670 | EID1 | EN5G00000255302 |
| LOC727930 | NC_000005.10 | DENR | EN5G00000139726 | PTPN12 | EN5G00000127947 |
| HS3ST3B1 | EN5G00000125430 | ZNF540 | EN5G00000171817 | SCARB2 | EN5G00000138760 |
| MEOX1 | ENSG00000005102 | TPTE2P1 | EN5G00000253771 | PROSC | EN5G00000147471 |
| E2F7 | EN5G00000165891 | POU3F1 | EN5G00000185668 | HSP90AA1 | EN5G00000080824 |
| AUTS2 | EN5G00000158321 | BOLL | EN5G00000152430 | HIPK3 | EN5G00000110422 |
| FUT4 | EN5G00000196371 | A2ML1 | EN5G00000166535 | AOX1 | EN5G00000138356 |
| DLX2 | EN5G00000115844 | SH2D4A | ENSG00000104611 | TJP1 | EN5G00000104067 |
| TBX3 | ENSG00000135111 | LOC100287525 | NC_000017.10 | PRNP | EN5G00000171867 |
| TIMP1 | EN5G00000102265 | SERPINB11 | EN5G00000206072 | ARL6IP5 | EN5G00000144746 |
| RAB31 | EN5G00000168461 | TBC1D10C | EN5G00000175463 | SQLE | EN5G00000104549 |
| CLIC4 | EN5G00000169504 | IL18BP | EN5G00000137496 | SEC23A | EN5G00000100934 |
| MXRA7 | EN5G00000182534 | GPR110 | EN5G00000153292 | FKBP1A | EN5G00000088832 |
| INPP5F | EN5G00000198825 | TPSAB1 | EN5G00000172236 | TNFSF18 | EN5G00000120337 |
| GEM | EN5G00000164949 | SSH2 | EN5G00000141298 | BCL2L1 | EN5G00000171552 |
| ANXA5 | ENSG00000164111 | C14orf109 | EN5G00000153485 | CREG1 | EN5G00000143162 |
| CST6 | EN5G00000175315 | DNAL1 | EN5G00000119661 | RIOK3 | EN5G00000101782 |
| RBPJ | EN5G00000168214 | NDP | EN5G00000124479 | PKN2 | EN5G00000065243 |
| PLAU | EN5G00000122861 | FGFRL1 | EN5G00000127418 | ST6GALNAC4 | EN5G00000136840 |
| ITGAV | EN5G00000138448 | XPO1 | EN5G00000082898 | IQGAP1 | EN5G00000140575 |
| MAP4 | ENSG00000047849 | MK167IP | ENSG00000155438 | TPR | ENSG00000047410 |
| ABHD2 | ENSG00000140526 | SLC7A1 | ENSG00000139514 | DAB2 | ENSG00000153071 |
| BMP1 | ENSG00000168487 | ALDH1A1 | ENSG00000165092 | ITGB4 | ENSG00000132470 |
| KCNK1 | ENSG00000135750 | PDXK | ENSG00000160209 | GNS | ENSG00000135677 |
| RGS16 | EN5G00000143333 | RDH12 | EN5G00000139988 | PPP1R2 | EN5G00000184203 |
| ATXN1 | ENSG00000124788 | DNMT3B | ENSG00000088305 | LIG4 | ENSG00000174405 |
| NMB | ENSG00000197696 | CYP2W1 | ENSG00000073067 | LYPLA1 | ENSG00000120992 |
| IL1RN | ENSG00000136689 | SYT2 | ENSG00000143858 | PAK2 | ENSG00000180370 |
| KLF10 | ENSG00000155090 | CLIP3 | ENSG00000105270 | CLDN1 | ENSG00000163347 |
| APOD | ENSG00000189058 | C9orf9 | ENSG00000165698 | RNF146 | ENSG00000118518 |
| PIK3IP1 | ENSG00000100100 | DDX28 | ENSG00000182810 | EIF5A | ENSG00000132507 |
| CRIP1 | ENSG00000213145 | NSFL1C | ENSG00000088833 | PTK2 | ENSG00000169398 |
| SLC5A3 | ENSG00000198743 | SPDEF | ENSG00000124664 | NBN | ENSG00000104320 |
| NR4A3 | ENSG00000119508 | AGAP3 | ENSG00000133612 | CPNE3 | ENSG00000085719 |
| EVI2A | ENSG00000126860 | CCDC123 | ENSG00000121289 | RAB14 | ENSG00000119396 |
| FAM102A | ENSG00000167106 | KLF12 | ENSG00000118922 | PANX1 | ENSG00000110218 |
| TIAM1 | ENSG00000156299 | SPINT1 | ENSG00000166145 | ARNTL | ENSG00000133794 |
| ALOX5AP | EN5G00000132965 | MIR155HG | EN5G00000234883 | TWSG1 | EN5G00000128791 |
| TNFSF4 | ENSG00000117586 | DDX3X | ENSG00000215301 | IGF2BP3 | ENSG00000136231 |
| PPAP2A | ENSG00000067113 | LOC158696 (or | ENSG00000129682 | PVR | ENSG00000073008 |
| | | FGF13) | | | |
| PKIA | ENSG00000171033 | ID3 | ENSG00000117318 | YY1 | ENSG00000100811 |
| FBXO11 | ENSG00000138081 | LOC728431 (or | ENSG00000233621 | PHTF2 | ENSG00000006576 |
| | | LINC01137) | | | |
| DCLRE1C | ENSG00000152457 | B3GNT7 | ENSG00000156966 | CDK17 | ENSG00000059758 |
| C18orf1 | ENSG00000168675 | MAB21L1 | ENSG00000180660 | HIST1H2BE | ENSG00000274290 |
| NPTX1 | ENSG00000171246 | TGIF1 | ENSG00000177426 | TBL1XR1 | ENSG00000177565 |
| CSGALNACT1 | ENSG00000147408 | FAM82A2 | ENSG00000137824 | BCAP29 | ENSG00000075790 |
| TMOD1 | ENSG00000136842 | LOC100133131 | NC_000004.10 | CSNK1A1 | ENSG00000113712 |
| ATP1B1 | EN5G00000143153 | RNFT1 | ENSG00000189050 | CBS | ENSG00000160200 |
| CCR4 | EN5G00000183813 | LCK | EN5G00000182866 | GLUD1 | EN5G00000148672 |
| CD83 | EN5G00000112149 | IL1A | ENSG00000115008 | SLC16A1 | EN5G00000155380 |
| LRIG1 | EN5G00000144749 | MXD1 | EN5G00000059728 | SPAG9 | EN5G00000008294 |
| ADO | EN5G00000181915 | REPS2 | EN5G00000169891 | PPFIBP1 | ENSG00000110841 |
| LRBA | EN5G00000198589 | C11orf75 | EN5G00000166002 | CREBL2 | EN5G00000111269 |
| DIXDC1 | EN5G00000150764 | MLPH | EN5G00000115648 | RYK | EN5G00000163785 |
| SYNJ2 | EN5G00000078269 | CCDC63 | EN5G00000173093 | MAX | EN5G00000125952 |
| RIMS3 | ENSG00000117016 | ENO3 | EN5G00000108515 | ARF4 | EN5G00000168374 |
| JUN | EN5G00000177606 | RABGAP1L | EN5G00000152061 | KIAA1109 | EN5G00000138688 |
| CD96 | ENSG00000153283 | SORL1 | ENSG00000137642 | B4GALT1 | ENSG00000086062 |
| PRKCD | EN5G00000163932 | PHACTR1 | ENSG00000112137 | TANK | EN5G00000136560 |
| S100PBP | EN5G00000116497 | ERBB3 | EN5G00000065361 | RAC2 | EN5G00000128340 |
| HLF | ENSG00000108924 | C11orf63 | ENSG00000109944 | TM9SF1 | ENSG00000100926 |
| ABCC4 | EN5G00000125257 | LOC731157 | EN5G00000255647 | IL8 | EN5G00000169429 |
| CXCL13 | ENSG00000156234 | CCDC114 | ENSG00000105479 | DSTYK | ENSG00000133059 |
| PPARG | ENSG00000132170 | CHERP | ENSG00000085872 | SCAMP1 | ENSG00000085365 |
| AHCYL2 | ENSG00000158467 | ROCK1 | ENSG00000067900 | PLD1 | ENSG00000075651 |
| AQP3 | ENSG00000165272 | CPEB3 | ENSG00000107864 | LUC7L3 | ENSG00000108848 |
| TSPAN13 | ENSG00000106537 | TYRO3 | ENSG00000092445 | PLAA | ENSG00000137055 |
| SLC1A4 | ENSG00000115902 | CRX | ENSG00000105392 | ARFGEF1 | ENSG00000066777 |
| TFEB | ENSG00000112561 | FARP1 | ENSG00000152767 | KDELR1 | ENSG00000105438 |
| ITGAE | ENSG00000083457 | ESR2 | ENSG00000140009 | CCPG1 | ENSG00000260916 |
| APOBEC3G | ENSG00000239713 | ISM1 | ENSG00000101230 | LAPTM4B | ENSG00000104341 |
| CDYL | ENSG00000153046 | UBE2E1 | ENSG00000170142 | GLUD2 | ENSG00000182890 |
| IL7R | ENSG00000168685 | BLID | ENSG00000259571 | SERPINB9 | ENSG00000170542 |
| IL9 | ENSG00000145839 | WNT4 | ENSG00000162552 | PTP4A1 | ENSG00000112245 |
| ZNF238 | EN5G00000179456 | PTPN22 | EN5G00000134242 | ATP2B1 | ENSG00000070961 |
| MYH6 | ENSG00000197616 | LOC285423 (or | ENSG00000234828 | HSPA13 | ENSG00000155304 |
| | | IQCM) | | | |
| GPR35 | ENSG00000178623 | LRPAP1 | ENSG00000163956 | WDFY3 | ENSG00000163625 |
| MB | ENSG00000198125 | TNS1 | ENSG00000079308 | TMCO3 | ENSG00000150403 |
| C5orf23 | ENSG00000113389 | RALGPS2 | ENSG00000116191 | IF127 | ENSG00000165949 |
| RAI14 | ENSG00000039560 | ADAMTS19 | ENSG00000145808 | POLG | ENSG00000140521 |
| AHNAK2 | ENSG00000185567 | C17orf66 | ENSG00000270379 | RALB | ENSG00000144118 |
| ZNF532 | ENSG00000074657 | SNAI3 | ENSG00000185669 | SOS2 | ENSG00000100485 |
| CEP170 | EN5G00000143702 | DSTN | EN5G00000125868 | OGT | EN5G00000147162 |
| POSTN | ENSG00000133110 | STOM | EN5G00000148175 | ITGA6 | EN5G00000091409 |
| PALM2-AKAP2 | EN5G00000157654 | KCNIP4 | EN5G00000185774 | GNB1 | EN5G00000078369 |
| SPP1 | EN5G00000118785 | TACC2 | EN5G00000138162 | CBFB | EN5G00000067955 |
| ERRFI1 | EN5G00000116285 | SLC25A30 | EN5G00000174032 | SPIN1 | EN5G00000106723 |
| APOE | EN5G00000130203 | CXorf31 | EN5G00000204904 | LMAN1 | EN5G00000074695 |
| PCOLCE2 | EN5G00000163710 | TP63 | EN5G00000073282 | PPPDE1 | EN5G00000121644 |
| EHD2 | EN5G00000024422 | SLC5A12 | EN5G00000148942 | UBA6 | EN5G00000033178 |
| ENO2 | EN5G00000111674 | RRAD | EN5G00000166592 | ATP2B4 | EN5G00000058668 |
| KCNJ8 | EN5G00000121361 | HAPLN1 | EN5G00000145681 | CDC73 | EN5G00000134371 |
| TANC2 | EN5G00000170921 | LTBR | ENSG00000111321 | WNK1 | EN5G00000060237 |
| VCAN | EN5G00000038427 | SYT14 | EN5G00000143469 | ZNF22 | EN5G00000165512 |
| CD109 | EN5G00000156535 | TNFRSF10D | EN5G00000173530 | LPP | EN5G00000145012 |
| CDKN1C | ENSG00000129757 | 3-Mar. | ENSG00000173926 | FLI1 | ENSG00000151702 |
| PLA2G16 | ENSG00000176485 | CCDC68 | ENSG00000166510 | NEDD4 | ENSG00000069869 |
| AXL | ENSG00000167601 | AK1 | ENSG00000106992 | PCDHGA3 | ENSG00000254245 |
| PLOD2 | ENSG00000152952 | CALHM3 | ENSG00000183128 | PPP1R12A | ENSG00000058272 |
| ULBP2 | ENSG00000131015 | MAP4K2 | ENSG00000168067 | PON2 | ENSG00000105854 |
| RASSF8 | ENSG00000123094 | MAP1LC3A | ENSG00000101460 | UBE2D3 | ENSG00000109332 |
| TBC1D8 | ENSG00000204634 | IL1R2 | ENSG00000115590 | TTC3 | ENSG00000182670 |
| SOX11 | ENSG00000176887 | KIAA0317 | ENSG00000119682 | MEIS3P1 | ENSG00000179277 |
| DOCK4 | ENSG00000128512 | SCGN | ENSG00000079689 | CSDA | ENSG00000060138 |
| SULF1 | ENSG00000137573 | CNTNAP3 | ENSG00000106714 | TMOD3 | ENSG00000138594 |
| IER5L | ENSG00000188483 | AGPAT2 | ENSG00000169692 | PTPN11 | ENSG00000179295 |
| C15orf52 | ENSG00000188549 | GNRHR | ENSG00000109163 | SH3BGRL | ENSG00000131171 |
| FGD6 | ENSG00000180263 | AGRP | ENSG00000159723 | EIF3A | ENSG00000107581 |
| PDLIM7 | ENSG00000196923 | HES1 | ENSG00000114315 | PHACTR2 | ENSG00000112419 |
| EBF1 | ENSG00000164330 | DMD | ENSG00000198947 | CRK | ENSG00000167193 |
| AMIGO2 | ENSG00000139211 | MTMR3 | ENSG00000100330 | UBE2J1 | ENSG00000198833 |
| PTPRM | ENSG00000173482 | EML4 | ENSG00000143924 | SDPR | ENSG00000168497 |
| BACH2 | ENSG00000112182 | DNASE1 | EN5G00000213918 | NCKAP1 | EN5G00000061676 |
| OLFML2B | EN5G00000162745 | PNKD | EN5G00000127838 | NFAT5 | ENSG00000102908 |
| MFGE8 | EN5G00000140545 | ACER3 | EN5G00000078124 | SHC1 | ENSG00000160691 |
| MYADM | ENSG00000179820 | NPAS1 | ENSG00000130751 | UBE2G1 | ENSG00000132388 |
| SLC16A6 | ENSG00000108932 | PSEN2 | ENSG00000143801 | RPRDIA | ENSG00000141425 |
| ACTN1 | ENSG00000072110 | MAPK8 | ENSG00000107643 | PPFIA1 | ENSG00000131626 |
| OLR1 | EN5G00000173391 | LOC553137 (or | EN5G00000235142 | NFATC2IP | EN5G00000176953 |
| | | LINC02532) | | | |
| DPP4 | ENSG00000197635 | UTRN | ENSG00000152818 | FGF2 | ENSG00000138685 |
| TRNP1 | ENSG00000253368 | BUB1 | ENSG00000169679 | KLHL7 | ENSG00000122550 |
| IGFBP4 | ENSG00000141753 | DCUN1D3 | ENSG00000188215 | SGPPI | ENSG00000126821 |
| B3GALNT1 | ENSG00000169255 | C8orf8 | ENSG00000236827 | HERC4 | ENSG00000148634 |
| PCDHB2 | ENSG00000112852 | CDH1 | ENSG00000039068 | MICAL2 | ENSG00000133816 |
| ABLIM3 | ENSG00000173210 | IGKC | ENSG00000211592 | SP110 | ENSG00000135899 |
| LMTK3 | ENSG00000142235 | EDNRB | ENSG00000136160 | SEC14L1 | ENSG00000129657 |
| HTRA1 | ENSG00000166033 | CDS1 | ENSG00000163624 | RAB5C | ENSG00000108774 |
| CLDN11 | EN5G00000013297 | GATA4 | EN5G00000136574 | RAD21 | EN5G00000164754 |
| TMCC3 | EN5G00000057704 | WIP11 | EN5G00000070540 | UTP3 | EN5G00000132467 |
| DPYSL3 | EN5G00000113657 | PHTF1 | EN5G00000116793 | GLIPR1 | EN5G00000139278 |
| ADAMTS2 | EN5G00000087116 | FTCD | EN5G00000160282 | NAA35 | EN5G00000135040 |
| LOC641518 | EN5G00000232021 | ZNF79 | EN5G00000196152 | NBR1 | EN5G00000188554 |
| MYH10 | EN5G00000133026 | SH3RF3 | EN5G00000172985 | TAF9B | EN5G00000187325 |
| SKI | EN5G00000157933 | C21orf67 | EN5G00000183250 | TOX4 | EN5G00000092203 |
| ELK3 | ENSG00000111145 | FLJ30375 (or | NC_000003.12 | LOXL2 | EN5G00000134013 |
| | | LOC440982) | | | |
| PHLDA1 | EN5G00000139289 | ZFP41 | EN5G00000181638 | EPB41L3 | EN5G00000082397 |
| CCDC88A | EN5G00000115355 | GLRX | EN5G00000173221 | NFIB | EN5G00000147862 |
| CYTH3 | EN5G00000008256 | C10orf95 | EN5G00000120055 | CALM1 | EN5G00000198668 |
| ATP10A | EN5G00000206190 | ID2B | NC_000003.12 | TMX1 | EN5G00000139921 |
| ITGA5 | EN5G00000161638 | LOC541473 | EN5G00000146722 | SIRPA | EN5G00000198053 |
| SLC43A3 | EN5G00000134802 | C6orf195 | EN5G00000164385 | DCTD | EN5G00000129187 |
| ZNF281 | EN5G00000162702 | FNDC5 | EN5G00000160097 | CHP | EN5G00000187446 |
| ADAM12 | EN5G00000148848 | WDR52 | EN5G00000206530 | NF1 | ENSG00000196712 |
| HSPG2 | EN5G00000142798 | SPINK5 | ENSG00000133710 | PAFAH1B1 | ENSG00000007168 |
| MSR1 | EN5G00000038945 | LOC642587 (or | ENSG00000230937 | ACBD3 | ENSG00000182827 |
| | | MIR205HG) | | | |
| LAMC1 | ENSG00000135862 | RGS12 | ENSG00000159788 | TFDP1 | ENSG00000198176 |
| APOC1 | ENSG00000130208 | TGM5 | ENSG00000104055 | FEM1B | ENSG00000169018 |
| VSIG10L | ENSG00000186806 | CYGB | ENSG00000161544 | HHLA3 | ENSG00000197568 |
| PACSIN3 | EN5G00000165912 | SYT5 | ENSG00000129990 | SYNM | ENSG00000182253 |
| LARP6 | ENSG00000166173 | LOC389634 (or | ENSG00000226091 | NFYA | ENSG00000001167 |
| | | LINC00937) | | | |
| THBS1 | ENSG00000137801 | LOC121952 (or | ENSG00000250878 | MGEA5 | ENSG00000198408 |
| | | METTL21EP) | | | |
| CXCR7 | ENSG00000144476 | DEFB124 | ENSG00000180383 | LYN | EN5G00000254087 |
| GABARAPL1 | ENSG00000139112 | CYB5A | ENSG00000166347 | KITLG | ENSG00000049130 |
| SLC11A1 | ENSG00000018280 | MYO5BP2 | EN5G00000238245 | CEACAM1 | EN5G00000079385 |
| BTG1 | ENSG00000133639 | P2RY11 | ENSG00000244165 | SNAP23 | ENSG00000092531 |
| MMP8 | ENSG00000118113 | RNF183 | ENSG00000165188 | TPP2 | ENSG00000134900 |
| SPHK1 | ENSG00000176170 | ABHD5 | ENSG00000011198 | NRAS | ENSG00000213281 |
| GDF5 | ENSG00000125965 | C15orf62 | ENSG00000188277 | ASF1A | ENSG00000111875 |
| HPGD | ENSG00000164120 | TRIM2 | ENSG00000109654 | XPO7 | ENSG00000130227 |
| KIAA1217 | ENSG00000120549 | LOC643837 (or | ENSG00000228794 | CDC5L | ENSG00000096401 |
| | | LINC01128) | | | |
| PECAM1 | ENSG00000261371 | PRSS21 | EN5G00000007038 | RASA1 | ENSG00000145715 |
| PDE4DIP | ENSG00000178104 | SLC6A4 | ENSG00000108576 | 7-Mar. | ENSG00000136536 |
| FLCN | ENSG00000154803 | TBC1D16 | ENSG00000167291 | IL13RA1 | ENSG00000131724 |
| THBS3 | ENSG00000169231 | ICK | ENSG00000112144 | ITPR3 | EN5G00000096433 |
| CXCR4 | ENSG00000121966 | OR51E1 | ENSG00000180785 | GLG1 | EN5G00000090863 |
| GPX3 | ENSG00000211445 | MINK1 | ENSG00000141503 | KPNA1 | ENSG00000114030 |
| TM6SF1 | EN5G00000136404 | ZNF800 | EN5G00000048405 | EXOC5 | EN5G00000070367 |
| DLC1 | EN5G00000164741 | OLIG2 | EN5G00000205927 | ABCF2 | EN5G00000033050 |
| FKBP1B | EN5G00000119782 | UNC13C | EN5G00000137766 | ASNS | EN5G00000070669 |
| S100A2 | ENSG00000196754 | RNF166 | ENSG00000158717 | HSP90B1 | ENSG00000166598 |
| BEAN | ENSG00000135094 | LOC729178 (or | ENSG00000233452 | ATG5 | ENSG00000057663 |
| | | STXBP5-AS1) | | | |
| NAV1 | EN5G00000134369 | ST18 | EN5G00000147488 | WAC | EN5G00000095787 |
| TREM1 | EN5G00000124731 | PPP2R2C | ENSG00000074211 | ATP6AP2 | EN5G00000182220 |
| PDK4 | ENSG00000004799 | GAD2 | ENSG00000136750 | FAIM | ENSG00000158234 |
| MEF2A | ENSG00000068305 | KCNK13 | ENSG00000152315 | ABI1 | ENSG00000136754 |
| ST3GAL6 | ENSG00000064225 | C14orf56 | NC_000014.8 | ADH5 | ENSG00000197894 |
| CDK14 | ENSG00000058091 | KLK2 | ENSG00000167751 | PWP1 | ENSG00000136045 |
| SPTBN1 | ENSG00000115306 | LRRC43 | ENSG00000158113 | ASPH | EN5G00000198363 |
| CAMK2N1 | ENSG00000162545 | TRAIP | ENSG00000183763 | DNAJC10 | ENSG00000077232 |
| RUNX2 | ENSG00000124813 | CACNA1E | ENSG00000198216 | SELT | ENSG00000198843 |
| SLIT3 | ENSG00000184347 | FAM71B | ENSG00000170613 | 2-Sep. | EN5G00000168385 |
| USP46 | EN5G00000109189 | BPY2 | EN5G00000183753 | ENO1 | EN5G00000074800 |
| LRRC32 | EN5G00000137507 | DBF4B | EN5G00000161692 | PTPRF | EN5G00000142949 |
| ANGPT2 | EN5G00000091879 | SYTL3 | EN5G00000164674 | CAPN7 | EN5G00000131375 |
| FADS3 | EN5G00000221968 | CYP2F1 | EN5G00000197446 | RBM9 | ENSG00000100320 |
| ADAM9 | EN5G00000168615 | OR1J4 | EN5G00000239590 | C5orf28 | EN5G00000151881 |
| CD151 | EN5G00000177697 | GBP6 | EN5G00000183347 | EIF4G1 | EN5G00000114867 |
| SLC44A2 | EN5G00000129353 | PLCG1 | EN5G00000124181 | HSF1 | EN5G00000185122 |
| GULP1 | EN5G00000144366 | LIPJ | EN5G00000204022 | HSPH1 | EN5G00000120694 |
| MID2 | EN5G00000080561 | TPD52 | EN5G00000076554 | NONO | EN5G00000147140 |
| LYNX1 | EN5G00000180155 | KLHL11 | EN5G00000178502 | IKBKB | EN5G00000104365 |
| COL22A1 | EN5G00000169436 | C9orf44 | EN5G00000225511 | TNFRSF10C | EN5G00000173535 |
| BNC2 | EN5G00000173068 | LRRC37BP1 | EN5G00000250462 | TMEM41B | EN5G00000166471 |
| SDCCAG8 | EN5G00000054282 | TMEM207 | EN5G00000198398 | PTPRO | EN5G00000151490 |
| ORA12 | EN5G00000160991 | TRIM36 | EN5G00000152503 | KPNA4 | EN5G00000186432 |
| MMP19 | EN5G00000123342 | SLCO2B1 | EN5G00000137491 | SPTLC1 | EN5G00000090054 |
| DENND3 | EN5G00000105339 | PPM1H | ENSG00000111110 | NRCAM | EN5G00000091129 |
| ZEB1 | EN5G00000148516 | CLDN23 | EN5G00000253958 | RAB5A | EN5G00000144566 |
| DNASE2 | EN5G00000105612 | TRPM3 | EN5G00000083067 | TCF25 | ENSG00000141002 |
| ADAM8 | EN5G00000151651 | AMBRA1 | EN5G00000110497 | NUCB1 | EN5G00000104805 |
| FST | EN5G00000134363 | SLC31A1 | EN5G00000136868 | RCN2 | EN5G00000117906 |
| IL1RAP | EN5G00000196083 | ASB15 | EN5G00000146809 | ZNF148 | EN5G00000163848 |
| AZ12 | EN5G00000163512 | NKG7 | EN5G00000105374 | LAMP1 | EN5G00000185896 |
| SOCS6 | EN5G00000170677 | C20orf7 | EN5G00000101247 | SMC3 | EN5G00000108055 |
| MRC2 | ENSG00000011028 | C12orf48 | EN5G00000185480 | SOS1 | EN5G00000115904 |
| UBXN2B | EN5G00000215114 | C18orf8 | EN5G00000141452 | GTF2I | EN5G00000263001 |
| HTRA4 | EN5G00000169495 | SNX7 | EN5G00000162627 | ITGA4 | EN5G00000115232 |
| SPOCD1 | ENSG00000134668 | LHX6 | ENSG00000106852 | RBM25 | ENSG00000119707 |
| ADARB1 | ENSG00000197381 | USP22 | ENSG00000124422 | USP34 | ENSG00000115464 |
| ANO4 | ENSG00000151572 | TLR3 | ENSG00000164342 | ANXA4 | ENSG00000196975 |
| DFNB31 | EN5G00000095397 | TASP1 | EN5G00000089123 | UBXN4 | EN5G00000144224 |
| RGS1 | ENSG00000090104 | SNX10 | ENSG00000086300 | ETS1 | ENSG00000134954 |
| SDS (or | EN5G00000166546 | SLC4A11 | EN5G00000088836 | DIAPH1 | ENSG00000131504 |
| BEAN1) | | | | | |
| DCLK1 | ENSG00000133083 | RAPGEF3 | ENSG00000079337 | SYPL1 | ENSG00000008282 |
| OBSL1 | ENSG00000124006 | TFCP2L1 | ENSG00000115112 | EXTL3 | ENSG00000012232 |
| TMEM180 | ENSG00000138111 | APOC4 | ENSG00000267467 | CALR | ENSG00000179218 |
| C21orf7 | ENSG00000156265 | HOXB8 | ENSG00000120068 | MGAT2 | ENSG00000168282 |
| GNG11 | EN5G00000127920 | NEB | ENSG00000183091 | CREB1 | ENSG00000118260 |
| CCL7 | ENSG00000108688 | QPCT | ENSG00000115828 | G3BP2 | ENSG00000138757 |
| UPP1 | ENSG00000183696 | SGCB | ENSG00000163069 | SLC4A7 | ENSG00000033867 |
| FZD4 | ENSG00000174804 | DUSP16 | ENSG00000111266 | KLHL9 | ENSG00000198642 |
| FCGR1B | ENSG00000198019 | ICOSLG | ENSG00000160223 | ELF1 | ENSG00000120690 |
| DPH3 | ENSG00000154813 | CAPN5 | ENSG00000149260 | YWHAB | ENSG00000166913 |
| N4BP2L2 | EN5G00000244754 | EHF | EN5G00000135373 | CCDC6 | ENSG00000108091 |
| GRAMD1B | ENSG00000023171 | ENOPH1 | ENSG00000145293 | SELE | ENSG00000007908 |
| GLI3 | ENSG00000106571 | DGAT2 | ENSG00000062282 | MFSD6 | ENSG00000151690 |
| FBLN5 | ENSG00000140092 | TIAM2 | ENSG00000146426 | SORBS3 | ENSG00000120896 |
| CLEC5A | ENSG00000258227 | INADL | ENSG00000132849 | CYP51A1 | ENSG00000001630 |
| MAMLD1 | ENSG00000013619 | N4BP2L1 | ENSG00000139597 | CD44 | ENSG00000026508 |
| TCEAL3 | ENSG00000196507 | LOC149773 (or | ENSG00000231290 | RAPGEF2 | ENSG00000109756 |
| | | APCDD1L-DT) | | | |
| SEMA4B | ENSG00000185033 | TFR2 | ENSG00000106327 | PIGC | ENSG00000135845 |
| IRS1 | ENSG00000169047 | HN1 | ENSG00000189159 | EDEM3 | ENSG00000116406 |
| GPC1 | ENSG00000063660 | SLC23A3 | ENSG00000213901 | ATP6V1A | ENSG00000114573 |
| WBP5 | ENSG00000185222 | LOC646576 (or | ENSG00000248890 | ZFR | ENSG00000056097 |
| | | HHIP-AS1) | | | |
| MYOZ1 | ENSG00000177791 | CYP27A1 | ENSG00000135929 | USP10 | ENSG00000103194 |
| FPR3 | ENSG00000187474 | K1AA1244 | ENSG00000112379 | WTAP | ENSG00000146457 |
| PROC | EN5G00000115718 | HPS4 | ENSG00000100099 | GDE1 | ENSG00000006007 |
| FNDC3B | EN5G00000075420 | PNPLA7 | EN5G00000130653 | DHX9 | EN5G00000135829 |
| FZD10 | EN5G00000111432 | CKLF | EN5G00000217555 | CDC27 | EN5G00000004897 |
| MRO | EN5G00000134042 | GPR125 | EN5G00000152990 | GFRA1 | EN5G00000151892 |
| ADM | EN5G00000148926 | PTH2R | EN5G00000144407 | TWF1 | EN5G00000151239 |
| PLXDC2 | EN5G00000120594 | ESR1 | EN5G00000091831 | STIP1 | EN5G00000168439 |
| MBD6 | EN5G00000166987 | PAQR5 | EN5G00000137819 | PSAT1 | EN5G00000135069 |
| LDLRAD3 | EN5G00000179241 | ENG | EN5G00000106991 | GMFB | EN5G00000197045 |
| SLC22A4 | ENSG00000197208 | DNAJC6 | ENSG00000116675 | RERE | ENSG00000142599 |
| SH2D3C | ENSG00000095370 | AACSL | ENSG00000250420 | COMT | ENSG00000093010 |
| KCNK10 | ENSG00000100433 | 0R4D2 | EN5G00000255713 | NUP50 | ENSG00000093000 |
| FRY | ENSG00000073910 | ATP2B2 | EN5G00000157087 | CD164 | EN5G00000135535 |
| RIN3 | ENSG00000100599 | PARP10 | ENSG00000178685 | TGOLN2 | ENSG00000152291 |
| MOAP1 | ENSG00000165943 | SLC24A1 | ENSG00000074621 | WIZ | ENSG00000011451 |
| TMEM223 | ENSG00000168569 | LFNG | ENSG00000106003 | ENC1 | ENSG00000171617 |
| D103 | EN5G00000197406 | CYP4Z2P | EN5G00000154198 | M6PR | ENSG00000003056 |
| TTC6 | ENSG00000139865 | ABCG1 | ENSG00000160179 | S100P | ENSG00000163993 |
| IL1RL1 | ENSG00000115602 | EPB41 | ENSG00000159023 | PGRMC1 | ENSG00000101856 |
| D1030S | ENSG00000258498 | SLC6A15 | ENSG00000072041 | RAD23A | ENSG00000179262 |
| CPE | ENSG00000109472 | PKD1L2 | ENSG00000166473 | NUDT4 | ENSG00000173598 |
| FAM89A | ENSG00000182118 | KLF14 | ENSG00000266265 | LIN7C | ENSG00000148943 |
| CD300LF | ENSG00000186074 | ARHGAP11A | ENSG00000198826 | RABEP1 | ENSG00000029725 |
| SLC29A1 | ENSG00000112759 | LOC441461 (or | ENSG00000255145 | VDAC1 | ENSG00000213585 |
| | | STX17-AS1) | | | |
| FMNL2 | ENSG00000157827 | RHEB | ENSG00000106615 | MED20 | ENSG00000124641 |
| MTUS1 | ENSG00000129422 | SRRM3 | ENSG00000177679 | SPRY2 | ENSG00000136158 |
| GRB10 | ENSG00000106070 | SERHL2 | ENSG00000183569 | SLC11A2 | ENSG00000110911 |
| FAM 110B | ENSG00000169122 | POPDC3 | ENSG00000132429 | SLC33A1 | ENSG00000169359 |
| LOC646903 | NC_000003.12 | APOL6 | ENSG00000221963 | SMAD5 | ENSG00000113658 |
| CRTAC1 | ENSG00000095713 | CLDN6 | ENSG00000184697 | BCLAF1 | ENSG00000029363 |
| C5orf62 | EN5G00000256235 | TBC1D2 | EN5G00000095383 | BUB3 | EN5G00000154473 |
| DOCK9 | ENSG00000088387 | GCG | ENSG00000115263 | NUS1P3 | ENSG00000234627 |
| TLR7 | ENSG00000196664 | NSUN7 | ENSG00000179299 | SENP3 | ENSG00000161956 |
| NMBR | ENSG00000135577 | C12orf37 | ENSG00000196243 | ZWILCH | ENSG00000174442 |
| ALOX5 | ENSG00000012779 | K1AA2026 | ENSG00000183354 | PSG6 | ENSG00000170848 |
| DNM3 | EN5G00000197959 | FANCC | EN5G00000158169 | MAPK1 | EN5G00000100030 |
| LHFPL2 | ENSG00000145685 | RSPO2 | ENSG00000147655 | MANEA | ENSG00000172469 |
| ITGA2 | ENSG00000164171 | CCR7 | ENSG00000126353 | SPAST | ENSG00000021574 |
| IFIT2 | ENSG00000119922 | SCCPDH | ENSG00000143653 | SNRNP27 | ENSG00000124380 |
| TGFBR1 | ENSG00000106799 | PSG7 | ENSG00000221878 | RNF115 | ENSG00000265491 |
| BTC | ENSG00000174808 | C14orf142 | ENSG00000170270 | TOR1AIP1 | ENSG00000143337 |
| TANC1 | ENSG00000115183 | IQCF3 | EN5G00000229972 | MED6 | EN5G00000133997 |
| CDK5RAP2 | ENSG00000136861 | CN5H6.4 | ENSG00000277232 | EPRS | ENSG00000136628 |
| FLRT2 | EN5G00000185070 | LPAR3 | EN5G00000171517 | VP541 | EN5G00000006715 |
| FAM27E3 | EN5G00000274026 | 5T14 | EN5G00000149418 | SMEK1 | EN5G00000100796 |
| MGC24103 | NC_000009.11 | 5T851A1 | EN5G00000111728 | YWHAE | EN5G00000108953 |
| SLC31A2 | EN5G00000136867 | FAM65C | EN5G00000042062 | SCYL2 | EN5G00000136021 |
| SPRY1 | EN5G00000164056 | CXXC4 | EN5G00000168772 | CTH | ENSG00000116761 |
| TNFRSF12A | ENSG00000006327 | ASGR1 | ENSG00000141505 | ACLY | ENSG00000131473 |
| RGL3 | ENSG00000205517 | ZAN | ENSG00000146839 | TNFRSF1OB | ENSG00000120889 |
| FHL3 | ENSG00000183386 | C1orf64 | ENSG00000183888 | PRKACB | ENSG00000142875 |
| LOC283033 (or | EN5G00000233395 | CNTN5 | EN5G00000149972 | GBX2 | EN5G00000168505 |
| LINC00841) | | | | | |
| LDB2 | ENSG00000169744 | OPALIN | ENSG00000197430 | MT1E | ENSG00000169715 |
| ATP11B | EN5G00000058063 | RASGEF1B | EN5G00000138670 | FBXW11 | EN5G00000072803 |
| DAPK1 | ENSG00000196730 | LOC100240726 | ENSG00000225849 | MT1X | ENSG00000187193 |
| | | (or MKRN7P) | | | |
| GAL3ST4 | ENSG00000197093 | SPATA2 | ENSG00000158480 | DDX18 | ENSG00000088205 |
| TMEM163 | EN5G00000152128 | SLC36A4 | EN5G00000180773 | TRRAP | EN5G00000196367 |
| ABCD1 | ENSG00000101986 | FBXL17 | ENSG00000145743 | CLPTM1 | ENSG00000104853 |
| LTB4R | EN5G00000213903 | SVEP1 | EN5G00000165124 | GRB2 | EN5G00000177885 |
| SLC6A8 | ENSG00000130821 | ATP9A | ENSG00000054793 | PAPOLA | ENSG00000090060 |
| MASP1 | EN5G00000127241 | PRAM1 | EN5G00000133246 | PREPL | EN5G00000138078 |
| TMEM51 | ENSG00000171729 | OGFOD1 | ENSG00000087263 | FNDC3A | ENSG00000102531 |
| XRCC6BP1 | EN5G00000166896 | CEACAM21 | ENSG00000007129 | MT1M | EN5G00000205364 |
| RNF17 | ENSG00000132972 | CIT | ENSG00000122966 | NFYB | ENSG00000120837 |
| LAIR1 | ENSG00000167613 | BMX | ENSG00000102010 | POT1 | ENSG00000128513 |
| ZNF618 | ENSG00000157657 | TAGAP | ENSG00000164691 | PRPF4 | ENSG00000136875 |
| ADAM17 | ENSG00000151694 | C10orf35 | ENSG00000171224 | GGA3 | ENSG00000125447 |
| DUSP1 | ENSG00000120129 | SH3BP4 | ENSG00000130147 | SNAPC3 | ENSG00000164975 |
| DHX58 | ENSG00000108771 | LOC339751 (or | ENSG00000238133 | SRSF2IP | ENSG00000139218 |
| | | MAP3K20-AS1) | | | |
| NCEH1 | EN5G00000144959 | MAPKAP1 | EN5G00000119487 | ABCB1 | EN5G00000085563 |
| SLC22A15 | ENSG00000163393 | KRT7 | ENSG00000135480 | CFLAR | ENSG00000003402 |
| NCOR2 | ENSG00000196498 | SAM D14 | ENSG00000167100 | K1AA0494 | ENSG00000159658 |
| ZNF555 | ENSG00000186300 | SPAG1 | ENSG00000104450 | C1orf144 | ENSG00000055070 |
| HOXC10 | ENSG00000180818 | GPRIN3 | ENSG00000185477 | PTMS | ENSG00000159335 |
| ARL9 | EN5G00000196503 | CHDH | ENSG00000016391 | KPNA6 | ENSG00000025800 |
| TSKU | ENSG00000182704 | CARNS1 | ENSG00000172508 | C6orf106 | ENSG00000196821 |
| SPATA12 | EN5G00000186451 | DENND1B | EN5G00000213047 | RPE | EN5G00000197713 |
| C1orf204 | ENSG00000188004 | FHAD1 | ENSG00000142621 | MAGT1 | ENSG00000102158 |
| CHD3 | ENSG00000170004 | BIN2 | ENSG00000110934 | SLC26A2 | ENSG00000155850 |
| IQCG | ENSG00000114473 | LOC100293679 | AC 000134.1 | ABCC1 | ENSG00000103222 |
| LOC100128501 | NC_000009.10 | KCNA2 | ENSG00000177301 | LRP8 | ENSG00000157193 |
| SLFN5 | ENSG00000166750 | ZNF81 | ENSG00000197779 | SYNCRIP | ENSG00000135316 |
| CCDC40 | ENSG00000141519 | TRA2A | EN5G00000164548 | PCBD1 | EN5G00000166228 |
| S100A11 | ENSG00000163191 | NCRNA00257 | ENSG00000255568 | SMARCC1 | ENSG00000173473 |
| MAPK8IP3 | ENSG00000138834 | TMEM144 | ENSG00000164124 | METAP2 | ENSG00000111142 |
| TM7SF4 | ENSG00000164935 | MS4Al2 | ENSG00000071203 | ANKFY1 | ENSG00000185722 |
| AICDA | ENSG00000111732 | KL | ENSG00000133116 | CLCN3 | ENSG00000109572 |
| PLEKHA7 | ENSG00000166689 | ITGA9 | ENSG00000144668 | SNX4 | ENSG00000114520 |
| C1orf106 | ENSG00000163362 | TNFSF13 | ENSG00000161955 | MBTPS1 | ENSG00000140943 |
| UNC5C | ENSG00000182168 | GPCPD1 | ENSG00000125772 | ALG13 | ENSG00000101901 |
| MYOZ3 | ENSG00000164591 | NCRNA00221 | ENSG00000270816 | GPR56 | ENSG00000205336 |
| ADCY2 | EN5G00000078295 | NUDT22 | EN5G00000149761 | FOXJ3 | EN5G00000198815 |
| KCNH3 | EN5G00000135519 | CHGB | EN5G00000089199 | MFNG | ENSG00000100060 |
| IL28RA | ENSG00000185436 | LOC440602 (or | ENSG00000235407 | AP1S1 | ENSG00000106367 |
| | | CYMP-AS1) | | | |
| JAG1 | ENSG00000101384 | SDSL | ENSG00000139410 | DCTN4 | ENSG00000132912 |
| MAP3K2 | ENSG00000169967 | ID2 | ENSG00000115738 | TSN | ENSG00000211460 |
| COL8A2 | ENSG00000171812 | PARD3B | ENSG00000116117 | TRA2B | EN5G00000136527 |
| TPST1 | ENSG00000169902 | LOC728705 | NC_000012.10 | RIPK1 | ENSG00000137275 |
| FGF7 | ENSG00000140285 | SLC9A5 | ENSG00000135740 | SERINC3 | ENSG00000132824 |
| DKK1 | ENSG00000107984 | PTGDR | ENSG00000168229 | GM2A | ENSG00000196743 |
| PRRG4 | ENSG00000135378 | SARDH | ENSG00000123453 | TAGLN2 | ENSG00000158710 |
| PPARD | ENSG00000112033 | CD1D | ENSG00000158473 | SMG1 | ENSG00000157106 |
| GPR157 | ENSG00000180758 | GRAMD4 | ENSG00000075240 | OSBP | ENSG00000110048 |
| AREG | ENSG00000109321 | LTB | EN5G00000227507 | MAN1A1 | ENSG00000111885 |
| FER1L4 | ENSG00000088340 | RGS6 | ENSG00000182732 | SH3GLB2 | ENSG00000148341 |
| UNC5B | ENSG00000107731 | ERN2 | EN5G00000134398 | FUCA1 | EN5G00000179163 |
| LOC100240734 | NC_000012.12 | CNR1 | EN5G00000118432 | APBA1 | EN5G00000107282 |
| RP1L1 | ENSG00000183638 | CRNDE | ENSG00000245694 | GSPT1 | ENSG00000103342 |
| NUPR1 | ENSG00000176046 | HIVEP3 | ENSG00000127124 | MCM4 | ENSG00000104738 |
| IQCH | ENSG00000103599 | TGM4 | ENSG00000163810 | H2AFY | ENSG00000113648 |
| WRNIP1 | ENSG00000124535 | ALDOB | ENSG00000136872 | RANBP2 | ENSG00000153201 |
| MCOLN3 | ENSG00000055732 | TPMT | ENSG00000137364 | HADHA | ENSG00000084754 |
| TTC7A | ENSG00000068724 | SLC27A2 | ENSG00000140284 | TLR4 | ENSG00000136869 |
| RAB23 | ENSG00000112210 | PTPRD | ENSG00000153707 | DAPK3 | ENSG00000167657 |
| PHF17 | ENSG00000077684 | CASZ1 | ENSG00000130940 | ELP3 | ENSG00000134014 |
| EVX1 | ENSG00000106038 | CAPN9 | ENSG00000135773 | SETD8 | ENSG00000183955 |
| FPR1 | ENSG00000171051 | ZC3H12B | ENSG00000102053 | ARFIP1 | EN5G00000164144 |
| AQP9 | ENSG00000103569 | LOC646482 | NC_000013.10 | ZFY | ENSG00000067646 |
| SIMI | ENSG00000112246 | SPIRE2 | ENSG00000204991 | C19orf6 | ENSG00000182087 |
| CACNA1G | EN5G00000006283 | SCML4 | EN5G00000146285 | TGFBR2 | EN5G00000163513 |
| NFKBIL2 | EN5G00000160949 | RNF213 | EN5G00000173821 | BSG | EN5G00000172270 |
| FAM84B | EN5G00000168672 | PRSS8 | EN5G00000052344 | NET02 | EN5G00000171208 |
| FAM7A3 | ENSG00000178081 | STRBP | EN5G00000165209 | MAPK14 | ENSG00000112062 |
| PEX14 | ENSG00000142655 | KLHL23 | ENSG00000213160 | ADD1 | ENSG00000087274 |
| MBOAT7 | EN5G00000125505 | OR1OH3 | EN5G00000171936 | FAS | ENSG00000026103 |
| GUCA1A | ENSG00000048545 | C14orf162 | ENSG00000267909 | CD46 | ENSG00000117335 |
| RUNX1 | ENSG00000159216 | MRPS12 | ENSG00000128626 | ARF3 | ENSG00000134287 |
| BEST1 | ENSG00000167995 | ITGB3 | ENSG00000259207 | HMGA1 | ENSG00000137309 |
| SPAM1 | ENSG00000106304 | ITGB1BP1 | ENSG00000119185 | AGFG1 | EN5G00000173744 |
| ADAMTS18 | ENSG00000140873 | WDHD1 | ENSG00000198554 | HNRNPC | ENSG00000092199 |
| HCFC1R1 | ENSG00000103145 | C22orf45 | ENSG00000178803 | MY09B | ENSG00000099331 |
| PPDX | ENSG00000143224 | FAM164A | ENSG00000104427 | YAP1 | ENSG00000137693 |
| STOX2 | ENSG00000173320 | IL4 | ENSG00000113520 | PCK2 | ENSG00000100889 |
| SPATS2L | ENSG00000196141 | SYDE1 | ENSG00000105137 | PRPF40A | ENSG00000196504 |
| TOP1P2 | NC_000022.11 | NUSAP1 | ENSG00000137804 | SEL1L | ENSG00000071537 |
| ARL4A | EN5G00000122644 | TAS2R4 | EN5G00000127364 | RAB6A | EN5G00000175582 |
| VSIG1 | ENSG00000101842 | ZNF451 | ENSG00000112200 | NOLC1 | ENSG00000166197 |
| P4HA2 | ENSG00000072682 | C17orf99 | ENSG00000187997 | MAPRE1 | ENSG00000101367 |
| KCP | EN5G00000135253 | C18orf62 | EN5G00000206026 | CDV3 | EN5G00000091527 |
| TRIM55 | EN5G00000147573 | VDR | ENSG00000111424 | SKAP2 | ENSG00000005020 |
| LOC283050 (or | EN5G00000224596 | PRR15 | EN5G00000176532 | ACTR2 | ENSG00000138071 |
| ZMIZ1-AS1) | | | | | |
| RHOB | ENSG00000143878 | PGP | ENSG00000184207 | ZC3H15 | ENSG00000065548 |
| DISP1 | EN5G00000154309 | ATP6V0A2 | EN5G00000185344 | ZMYND11 | ENSG00000015171 |
| MMP14 | ENSG00000157227 | DHDH | ENSG00000104808 | ITPR2 | ENSG00000123104 |
| MMP7 | ENSG00000137673 | TRAF3IP2 | ENSG00000056972 | DAZAP2 | ENSG00000183283 |
| ABCG2 | EN5G00000118777 | MAST3 | EN5G00000099308 | EDC3 | ENSG00000179151 |
| ABHD8 | EN5G00000127220 | ERLIN1 | EN5G00000107566 | PCY0X1 | ENSG00000116005 |
| ARL5A | ENSG00000162980 | C6orf142 | ENSG00000146147 | MAP2K2 | ENSG00000126934 |
| GOS2 | ENSG00000123689 | TNXB | ENSG00000168477 | TFG | ENSG00000114354 |
| CMTM1 | ENSG00000089505 | TRIM58 | ENSG00000162722 | KCTD20 | ENSG00000112078 |
| FFAR1 | ENSG00000126266 | KIAA1543 (or | ENSG00000076826 | MTMR1 | ENSG00000063601 |
| | | CAMSAP3) | | | |
| MYH8 | ENSG00000133020 | SLC22A5 | ENSG00000197375 | CAPRIN1 | ENSG00000135387 |
| TES | ENSG00000135269 | LOC151121 (or | ENSG00000204460 | PIP5K1A | ENSG00000143398 |
| | | LINC01854) | | | |
| SEMA6B | ENSG00000167680 | LOC553103 (or | ENSG00000233006 | HNRNPH1 | ENSG00000169045 |
| | | MIR3936HG) | | | |
| CES1 | ENSG00000198848 | K1F23 | ENSG00000137807 | C14orf101 | ENSG00000070269 |
| SLC10A2 | ENSG00000125255 | TNFSF14 | ENSG00000125735 | TMED2 | ENSG00000086598 |
| KLHL6 | ENSG00000172578 | IL18R1 | ENSG00000115604 | CLN5 | ENSG00000102805 |
| LAPTM5 | ENSG00000162511 | PDE4B | ENSG00000184588 | GRLF1 | ENSG00000160007 |
| CLDN12 | ENSG00000157224 | GKAP1 | ENSG00000165113 | MPZL1 | ENSG00000197965 |
| GPR26 | ENSG00000154478 | LOC285045 (or | ENSG00000230876 | ROD1 | ENSG00000119314 |
| | | LINC00486) | | | |
| LOC151438 (or | EN5G00000281162 | AKR1C3 | EN5G00000196139 | AP2B1 | ENSG00000006125 |
| LINC01127) | | | | | |
| C1orf150 | ENSG00000169224 | VPS45 | ENSG00000136631 | PRKDC | ENSG00000253729 |
| PEX2 | ENSG00000164751 | VWF | ENSG00000110799 | SEC23IP | ENSG00000107651 |
| FAM113B | EN5G00000179715 | GH1 | EN5G00000259384 | RRN3P1 | EN5G00000248124 |
| FABP6 | ENSG00000170231 | CCDC129 | EN5G00000180347 | NUMA1 | EN5G00000137497 |
| SNTB1 | ENSG00000172164 | STEAP3 | ENSG00000115107 | GDI2 | ENSG00000057608 |
| LIMS1 | ENSG00000169756 | TSPAN17 | ENSG00000048140 | ME2 | ENSG00000082212 |
| KLF15 | ENSG00000163884 | PM20D2 | ENSG00000146281 | CYB5R4 | ENSG00000065615 |
| SOAT1 | EN5G00000057252 | PKHD1L1 | EN5G00000205038 | NAA15 | EN5G00000164134 |
| SH2D1B | EN5G00000198574 | ZNF608 | EN5G00000168916 | NNT | EN5G00000112992 |
| RNASEH2B | ENSG00000136104 | SOX1 | ENSG00000182968 | TRIM27 | ENSG00000204713 |
| MET | ENSG00000105976 | HMGB3 | ENSG00000029993 | DLG1 | ENSG00000075711 |
| SLC39Al2 | ENSG00000148482 | MYOZ2 | ENSG00000172399 | IGF2R | ENSG00000197081 |
| KRT13 | ENSG00000171401 | FU37453 (or | EN5G00000146722 | GTF2F1 | EN5G00000125651 |
| | | LOC541473) | | | |
| RAB3B | ENSG00000169213 | ATOH8 | ENSG00000168874 | TUBGCP2 | ENSG00000130640 |
| MAN2A2 | ENSG00000196547 | ERGIC1 | ENSG00000113719 | ERC1 | ENSG00000082805 |
| CNBD1 | ENSG00000176571 | SERPINA1 | ENSG00000197249 | COPA | ENSG00000122218 |
| VASH1 | EN5G00000071246 | ATP8B1 | EN5G00000081923 | DLAT | EN5G00000150768 |
| SEC22C | EN5G00000093183 | KRT222 | EN5G00000213424 | SCP2 | ENSG00000116171 |
| UBQLNL | ENSG00000175518 | IL1B | ENSG00000125538 | MAGOH | ENSG00000162385 |
| SUCLG1 | ENSG00000163541 | FAM195A | ENSG00000172366 | SRP72 | ENSG00000174780 |
| ZFYVE19 | ENSG00000166140 | VRK3 | ENSG00000105053 | TLE4 | ENSG00000106829 |
| RIF1 | EN5G00000080345 | MRE11A | EN5G00000020922 | MAT2A | EN5G00000168906 |
| RHOBTB2 | ENSG00000008853 | C18orf16 | ENSG00000260372 | UBE4B | ENSG00000130939 |
| UNC80 | ENSG00000144406 | CNTNAP2 | ENSG00000174469 | FAM120A | ENSG00000048828 |
| ARMC8 | ENSG00000114098 | NEK11 | ENSG00000114670 | SOCS2 | ENSG00000120833 |
| CADM2 | ENSG00000175161 | EDN3 | ENSG00000124205 | CANX | ENSG00000127022 |
| C3 | ENSG00000125730 | HNF4A | ENSG00000101076 | SSR1 | ENSG00000124783 |
| SLC8A1 | ENSG00000183023 | GLTSCR2 | ENSG00000105373 | AASDHPPT | ENSG00000149313 |
| ABCC3 | EN5G00000108846 | ARID5B | EN5G00000150347 | API5 | ENSG00000166181 |
| DPY19L1 | EN5G00000173852 | DAB2IP | EN5G00000136848 | SLC30A1 | EN5G00000170385 |
| TSPAN5 | EN5G00000168785 | TRIM50 | EN5G00000146755 | GGA2 | EN5G00000103365 |
| AAA1 (or | NC_001136.10 | PNPLA3 | EN5G00000100344 | VAC14 | EN5G00000103043 |
| NAT1) | | | | | |
| SLC26A10 | EN5G00000135502 | LOC389023 (or | EN5G00000235026 | KPNA3 | EN5G00000102753 |
| | | DPP10-AS1) | | | |
| HAMP | ENSG00000105697 | BCL2L2 | ENSG00000129473 | TTC37 | ENSG00000198677 |
| MERTK | ENSG00000153208 | DKFZp566F0947 | NC_000019.9 | SSB | ENSG00000138385 |
| | | (or LOC94023) | | | |
| TRIM62 | ENSG00000116525 | MAOA | ENSG00000189221 | C1orf103 | ENSG00000121931 |
| TP53111 | EN5G00000175274 | H2AFY2 | EN5G00000099284 | ARF1 | EN5G00000143761 |
| CLDND2 | ENSG00000160318 | HRK | ENSG00000135116 | 6-Mar. | ENSG00000145495 |
| SLC16A10 | ENSG00000112394 | SYNPR | ENSG00000163630 | RELN | ENSG00000189056 |
| AMPH | ENSG00000078053 | BSND | ENSG00000162399 | ARHGDIA | ENSG00000141522 |
| ZNF687 | ENSG00000143373 | GPD1 | ENSG00000167588 | C20orf30 | ENSG00000089063 |
| CNTNAP3B | EN5G00000154529 | ALX4 | EN5G00000052850 | PNO1 | EN5G00000115946 |
| TPM1 | ENSG00000140416 | CRMP1 | ENSG00000072832 | ADD3 | ENSG00000148700 |
| GPX7 | ENSG00000116157 | SLC25A37 | ENSG00000147454 | TFRC | ENSG00000072274 |
| NCRNA00113 | ENSG00000225298 | PRY | ENSG00000169789 | PRPF4B | ENSG00000112739 |
| MTHFD1L | ENSG00000120254 | PTK2B | ENSG00000120899 | RAB1A | ENSG00000138069 |
| NOS3 | EN5G00000164867 | LOC340017 (or | ENSG00000234111 | RHOBTB3 | EN5G00000164292 |
| | | LINC02433) | | | |
| LOC286437 | NC_000023.11 | CAMK4 | ENSG00000152495 | ZNF192 | ENSG00000198315 |
| SLC24A2 | EN5G00000155886 | FANCI | EN5G00000140525 | CHD4 | ENSG00000111642 |
| RNF6 | ENSG00000127870 | C15orf48 | ENSG00000166920 | ElF1AX | ENSG00000173674 |
| SERINC2 | ENSG00000168528 | ATP9B | ENSG00000166377 | DEDD | ENSG00000158796 |
| PSME4 | ENSG00000068878 | ADAM3A | ENSG00000197475 | MOBKL1B | ENSG00000114978 |
| C8orf55 | ENSG00000130193 | HOXA3 | ENSG00000105997 | TP53 | ENSG00000141510 |
| TSPAN3 | ENSG00000140391 | GNA14 | ENSG00000156049 | TMBIM6 | ENSG00000139644 |
| CADM1 | ENSG00000182985 | MPP7 | ENSG00000150054 | HPCAL1 | ENSG00000115756 |
| SLC16A3 | ENSG00000141526 | MBNL1 | ENSG00000152601 | ANAPC5 | ENSG00000089053 |
| LOC154822 (or | ENSG00000231419 | FLJ90680 | NC_000022.10 | STX16 | ENSG00000124222 |
| LINC00689) | | | | | |
| TFAP2C | ENSG00000087510 | C11orf93 | EN5G00000214290 | E1F4E | EN5G00000151247 |
| FAM4OB | EN5G00000128578 | SLC34A1 | ENSG00000131183 | MBTPS2 | ENSG00000012174 |
| 1L3RA | ENSG00000185291 | TMEM185A | ENSG00000269556 | COMM D10 | ENSG00000145781 |
| 1F1T3 | ENSG00000119917 | C6orf182 | ENSG00000183137 | SRSF10 | ENSG00000188529 |
| SH3GL2 | EN5G00000107295 | KRT38 | ENSG00000171360 | PSG3 | EN5G00000221826 |
| GLCCI1 | ENSG00000106415 | PAPLN | ENSG00000100767 | GLYR1 | EN5G00000140632 |
| KRTAP11-1 | EN5G00000182591 | SEMA6A | EN5G00000092421 | STS | ENSG00000101846 |
| C1orf91 | ENSG00000160055 | HS6ST3 | ENSG00000185352 | TIA1 | ENSG00000116001 |
| EPAS1 | ENSG00000116016 | C5orf56 | ENSG00000197536 | ANP32A | ENSG00000140350 |
| OR10D3 | ENSG00000197309 | BRSK1 | ENSG00000160469 | SYNJ1 | ENSG00000159082 |
| ASIP | ENSG00000101440 | APCDD1 | EN5G00000154856 | KCTD5 | EN5G00000167977 |
| TDRD9 | ENSG00000156414 | SLC35F1 | ENSG00000196376 | NFYC | ENSG00000066136 |
| YPEL2 | ENSG00000175155 | CCDC125 | ENSG00000183323 | LARP4B | ENSG00000107929 |
| MIA3 | EN5G00000154305 | FDX1 | EN5G00000137714 | KDM6A | ENSG00000147050 |
| BTBD19 | ENSG00000222009 | RFFL | ENSG00000092871 | POLR2E | ENSG00000099817 |
| GPR84 | EN5G00000139572 | 5LC25A35 | EN5G00000125434 | WDR1 | ENSG00000071127 |
| PHC2 | EN5G00000134686 | ANP32C | EN5G00000248546 | CCT2 | EN5G00000166226 |
| MGST1 | EN5G00000008394 | LOC100131733 | EN5G00000256262 | STX3 | ENSG00000166900 |
| | | (or USP30-AS1) | | | |
| MFAP3L | ENSG00000198948 | AP0A2 | ENSG00000158874 | AMD1 | ENSG00000123505 |
| NR4A2 | EN5G00000153234 | ID1 | EN5G00000125968 | PDE8A | EN5G00000073417 |
| GLUL | ENSG00000135821 | SV2B | ENSG00000185518 | U2AF2 | ENSG00000063244 |
| TLE3 | ENSG00000140332 | GALNT2 | ENSG00000143641 | BRCC3 | ENSG00000185515 |
| MAPKAPK2 | ENSG00000162889 | LOC652993 | AC_000144.1 | SERBP1 | ENSG00000142864 |
| DDA1 | ENSG00000130311 | ZNF552 | ENSG00000178935 | KIF5B | ENSG00000170759 |
| TPD52L1 | ENSG00000111907 | TRAC | ENSG00000277734 | HSPD1 | ENSG00000144381 |
| FOXD4 | ENSG00000170122 | TOP2A | ENSG00000131747 | POLDIP3 | ENSG00000100227 |
| GNB4 | ENSG00000114450 | LYPD6 | ENSG00000187123 | YKT6 | ENSG00000106636 |
| LSR | ENSG00000105699 | IQGAP3 | ENSG00000183856 | UBE3B | ENSG00000151148 |
| SMURF1 | ENSG00000198742 | ZNF627 | ENSG00000198551 | SEC63 | ENSG00000025796 |
| C2orf40 | ENSG00000119147 | CDH23 | ENSG00000107736 | G3BP1 | ENSG00000145907 |
| FKRP | ENSG00000181027 | FNBP1L | ENSG00000137942 | ILF3 | ENSG00000129351 |
| CLCN4 | EN5G00000073464 | PDE1B | EN5G00000123360 | CYB5B | ENSG00000103018 |
| OSM | ENSG00000099985 | LOC645638 (or | ENSG00000261040 | ROCK2 | ENSG00000134318 |
| | | WFDC21P) | | | |
| HECW1 | EN5G00000002746 | NKD1 | ENSG00000140807 | PTBP1 | ENSG00000011304 |
| DIRAS2 | EN5G00000165023 | PPP1R9A | EN5G00000158528 | K1AA0776 | ENSG00000014123 |
| CLVS1 | ENSG00000177182 | PSPH | ENSG00000146733 | AGA | ENSG00000038002 |
| MME | EN5G00000196549 | GABRA1 | EN5G00000022355 | GATC | EN5G00000257218 |
| ZNF549 | ENSG00000121406 | C9orf68 | ENSG00000106686 | GPR137 | ENSG00000173264 |
| DEC1 | ENSG00000173077 | GLTPD2 | ENSG00000182327 | GFPT1 | ENSG00000198380 |
| SS18 | N5G00000141380 | SAMD11 | EN5G00000187634 | STAM2 | ENSG00000115145 |
| DGCR6L | ENSG00000128185 | SLC35D1 | ENSG00000116704 | SMPD1 | ENSG00000166311 |
| RWDD2A | ENSG00000013392 | CEACAM7 | ENSG00000007306 | MARCKS | ENSG00000277443 |
| MBD4 | ENSG00000129071 | MAPK13 | ENSG00000156711 | UBE2G2 | ENSG00000184787 |
| RUNX1T1 | ENSG00000079102 | GAS2L1 | ENSG00000185340 | EPB41L2 | ENSG00000079819 |
| PXN | ENSG00000089159 | ASF1B | ENSG00000105011 | AKIRIN1 | ENSG00000174574 |
| LOC283104 (or | ENSG00000246273 | TCF21 | ENSG00000118526 | MCM3AP | ENSG00000160294 |
| SBF2-AS1) | | | | | |
| ECE1 | EN5G00000117298 | APBB1IP | EN5G00000077420 | GART | ENSG00000159131 |
| FZD7 | EN5G00000155760 | CXCL14 | EN5G00000145824 | ESF1 | EN5G00000089048 |
| RNASE4 | EN5G00000258818 | CCDC67 | EN5G00000165325 | CLINT1 | EN5G00000113282 |
| PRIM2 | EN5G00000146143 | HES5 | EN5G00000197921 | SRPR | EN5G00000182934 |
| BTBD11 | ENSG00000151136 | BLM | ENSG00000197299 | SEL1L3 | ENSG00000091490 |
| TLX3 | ENSG00000164438 | TMEM54 | ENSG00000121900 | SLC35A2 | ENSG00000102100 |
| HP | ENSG00000257017 | STOX1 | ENSG00000165730 | SDHC | ENSG00000143252 |
| ZNF589 | ENSG00000164048 | XYLT1 | ENSG00000103489 | MMP1 | ENSG00000196611 |
| TRIO | ENSG00000038382 | CELSR2 | ENSG00000143126 | AGPS | ENSG00000018510 |
| PRPF4OB | ENSG00000110844 | CDK1 | ENSG00000170312 | PLXNA2 | ENSG00000076356 |
| SOX18 | ENSG00000203883 | CXCR6 | ENSG00000172215 | HNRNPR | ENSG00000125944 |
| DDHD1 | ENSG00000100523 | SEMA3C | ENSG00000075223 | GRAP | ENSG00000154016 |
| CLPS | ENSG00000137392 | ZNF124 | ENSG00000196418 | SRRT | ENSG00000087087 |
| ERMP1 | ENSG00000099219 | TIAL1 | ENSG00000151923 | KIAA0182 | ENSG00000131149 |
| SLC16A8 | ENSG00000100156 | RREB1 | ENSG00000124782 | C6orf62 | ENSG00000112308 |
| CDKN1A | ENSG00000124762 | APOB48R | ENSG00000184730 | ORC5L | ENSG00000164815 |
| AK7 | ENSG00000140057 | HSD11B2 | EN5G00000176387 | NUDT21 | ENSG00000167005 |
| STX6 | EN5G00000135823 | CENPA | ENSG00000115163 | LSS | EN5G00000160285 |
| PHYHD1 | ENSG00000175287 | LOC100289219 | ENSG00000163956 | CTSS | ENSG00000163131 |
| | | (or LRPAP1) | | | |
| PCBP3 | EN5G00000183570 | AQP11 | ENSG00000178301 | PDCD4 | EN5G00000150593 |
| OLFML3 | EN5G00000116774 | GRAP2 | ENSG00000100351 | TBX1 | EN5G00000184058 |
| ZBTB16 | ENSG00000109906 | CDC25A | EN5G00000164045 | RNF14 | ENSG00000013561 |
| C9orf117 | ENSG00000160401 | FAM84A | ENSG00000162981 | DIMT1L | ENSG00000086189 |
| PCDHGB8P | ENSG00000248449 | PRLR | ENSG00000113494 | PLEKHB2 | ENSG00000115762 |
| EYA2 | ENSG00000064655 | BTNL8 | ENSG00000113303 | SRSF1 | ENSG00000136450 |
| TMTC1 | ENSG00000133687 | WNT5A | ENSG00000114251 | CSTF2T | ENSG00000177613 |
| EN1 | ENSG00000163064 | CEBPA | ENSG00000245848 | HS2ST1 | ENSG00000153936 |
| ELL2 | ENSG00000118985 | NCRNA00265 | ENSG00000188185 | CSNK2A1 | ENSG00000101266 |
| SLC45A3 | ENSG00000158715 | ARHGEF19 | ENSG00000142632 | TOP1 | ENSG00000198900 |
| HRG | ENSG00000113905 | PTTG1 | ENSG00000164611 | PTPRB | EN5G00000127329 |
| DENND5B | EN5G00000170456 | ACVR1B | EN5G00000135503 | LSM12 | EN5G00000161654 |
| MDFIC | EN5G00000135272 | SAE1 | EN5G00000142230 | USP1 | EN5G00000162607 |
| TGM2 | EN5G00000198959 | MAEA | EN5G00000090316 | ZFPL1 | EN5G00000162300 |
| GPD2 | EN5G00000115159 | PPIF | EN5G00000108179 | TSPAN12 | EN5G00000106025 |
| AKR1C2 | EN5G00000151632 | ALDH2 | EN5G00000111275 | 5LC35A3 | EN5G00000117620 |
| GLDN | EN5G00000186417 | AGMAT | EN5G00000116771 | UBE2K | EN5G00000078140 |
| OCIAD2 | EN5G00000145247 | FAM46C | EN5G00000183508 | CCND2 | EN5G00000118971 |
| LONRF3 | EN5G00000175556 | PAPOLB | EN5G00000218823 | DSCR3 | EN5G00000157538 |
| MAP9 | EN5G00000164114 | ASRGL1 | EN5G00000162174 | GCLM | EN5G00000023909 |
| LOC283454 (or | EN5G00000135116 | ASAP1 | EN5G00000153317 | PAPSS2 | EN5G00000198682 |
| HRK) | | | | | |
| NTRK2 | EN5G00000148053 | SAV1 | EN5G00000151748 | SRPK1 | EN5G00000096063 |
| SLC6Al2 | ENSG00000111181 | WWTR1 | ENSG00000018408 | GOT1 | ENSG00000120053 |
| IL18RAP | ENSG00000115607 | ARL4C | ENSG00000188042 | ACOT7 | ENSG00000097021 |
| PLEKHA5 | EN5G00000052126 | NRP1 | EN5G00000099250 | DCAF7 | EN5G00000136485 |
| NT5DC4 | ENSG00000144130 | BNIP3L | EN5G00000104765 | IDH3A | ENSG00000166411 |
| CPD | ENSG00000108582 | RBMS1 | ENSG00000153250 | PRPF6 | ENSG00000101161 |
| ANKRD29 | ENSG00000154065 | MYOF | ENSG00000138119 | YBX1 | ENSG00000065978 |
| PCMTD1 | ENSG00000168300 | TGFB1 | ENSG00000105329 | UBE2N | ENSG00000177889 |
| FAM178B | ENSG00000168754 | HIP1 | ENSG00000127946 | HSPA9 | ENSG00000113013 |
| ZFP36L1 | ENSG00000185650 | CDH2 | ENSG00000170558 | LARP4 | ENSG00000161813 |
| SMURF2 | ENSG00000108854 | UGCG | ENSG00000148154 | CYP20A1 | ENSG00000119004 |
| SIGLEC8 | ENSG00000105366 | IDS | ENSG00000010404 | EPHB2 | ENSG00000133216 |
| LOC284454 | NC_000019.10 | RNF13 | ENSG00000082996 | TFAM | ENSG00000108064 |

1. A method of treating cancer patients comprising the steps of:
a) determining whether the patient is expressing high levels of TGF-β response signatures in cells that comprise the tumor microenvironment (TME); and
b) if the patient has high levels of TGF-β response signatures in fibroblasts, then co-administering TGF-β inhibitor with human immunoglobulin.

2. The method according to claim 1, wherein the TME is selected from the group comprising fibroblasts, T-cells, macrophages or endothelial cells.

3. The method according to claim 1, wherein the cancer is selected from colorectal cancer, melanoma, breast cancer, bladder cancer, colon cancer, kidney cancer, lung cancer, ovary cancer, pancreas cancer, prostate cancer, rectal cancer, stomach cancer, thyroid cancer, uterus cancer and other types of cancer.

4. The cancer according to claim 2, wherein the cancer is colorectal cancer.

5. The method according to claim 1, wherein the TGF-β response signature is F-TBRS, T-TBRS, M-TBRS or E-TBRS.

6. The method according to claim 1, wherein the TGF-β response signature Vactosertib TGF-β response signature (VRS) comprising SERPINE1(PAI1), GADD45B, TIMP3, LMCD1, PLAUR, IL6, NUAK1, DACT1, EPHA4, SNAI1, and MEOX1.

7. The method according to claim 1, wherein the gene expression levels of TGF-β response signature in fibroblasts is determined by a Stromal Score analyzed by histopathological image of sample obtained from the patient.

8. The method according to claim 5, wherein the Stromal Score value of between 0 and 1 indicates the high TGF-β response signature level.

9. The method according to claim 1, wherein the gene expression levels of TGF-β response signature in fibroblasts is determined by RNA sequence analysis obtained from the patient.

10. The method according to claim 1, wherein the treatment is co-administering TGF-β inhibitor with human immunoglobulin.

11. The method according to claim 8, wherein TGF-β inhibitor is TEW-7197.

12. The method according to claim 8, wherein human immunoglobulin is selected from Pembrolizumab or Durvalumab.

13. A method for selecting the cancer patient which is likely to benefit from adjuvant therapy comprising the determination of the gene expression levels of TGF-β response signature,
wherein an increased expression level of said gene with respect to a reference value for said gene is indicative that the patient is likely to benefit from said therapy or
wherein a decreased expression level of said gene with respect to a reference value for said gene is indicative that the patient is unlikely to benefit from said therapy.

14. The method according to claim 11, wherein the TGF-β response signature is F-TBRS, T-TBRS, M-TBRS or E-TBRS.

15. The method according to claim 11, wherein the TGF-β response signature is Vactosertib TGF-β response signature (VRS) comprising SERPINE1(PAI1), GADD45B, TIMP3, LMCD1, PLAUR, IL6, NUAK1, DACT1, EPHA4, SNAI1, and MEOX1.

16. The method according to claim 11, wherein the expression levels of TGF-β response signature is determined by the Stromal Score analyzed by histopathological image of sample obtained from the patient.

17. The method according to claim 11, wherein increased expression level is defined as a stromal score between 0 and 1.

18. The method according to claim 11, wherein decreased expression level is defined as a stromal score between −1 and 0.

19. The method according to claim 11, wherein the therapy is co-administering TGF-β inhibitor with human immunoglobulin.

20. The method according to claim 8, wherein TGF-β inhibitor is TEW-7197.

21. The method according to claim 8, wherein human immunoglobulin is selected from Pembrolizumab or Durvalumab.
